SEQUENCE LISTING (11 sequences)

SEQ ID NO: 1 - protein, 810 aa, Homo sapiens
MPMDLILVVWFCVCTARTVVGFGMDPDLQMDIVTELDLVNTTLGVAQVSGMHNASKAFLFQDIEREIHAAPHVSEKLIQLFQNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCSGDCPHEGGLKHNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNITYDIRKTCLDSYGVSRLSGSVWTMAGSPCTTCKCKNGRVCCSVDFECLQNN

SEQ ID NO: 2 - protein, 810 aa, Mus musculus
MPMDVILVLWFCVCTARTVLGFGMDPDLQMDIITELDLVNTTLGVTQVAGLHNASKAFLFQDVQREIHSAPHVSEKLIQLFRNKSEFTFLATVQQKPSTSGVILSIRELEHSYFELESSGPREEIRYHYIHGGKPRTEALPYRMADGQWHKVALSVSASHLLLHVDCNRIYERVIDPPETNLPPGSNLWLGQRNQKHGFFKGIIQDGKIIFMPNGFITQCPNLNRTCPTCSDFLSLVQGIMDLQELLAKMTAKLNYAETRLGQLENCHCEKTCQVSGLLYRDQDSWVDGDNCRNCTCKSGAVECRRMSCPPLNCSPDSLPVHISGQCCKVCRPKCIYGGKVLAEGQRILTKTCRECRGGVLVKITEACPPLNCSEKDHILPENQCCRVCRGHNFCAEAPKCGENSECKNWNTKATCECKNGYISVQGNSAYCEDIDECAAKMHYCHANTVCVNLPGLYRCDCIPGYIRVDDFSCTEHDDCGSGQHNCDKNAICTNTVQGHSCTCQPGYVGNGTVCKAFCEEGCRYGGTCVAPNKCVCPSGFTGSHCEKDIDECAEGFVECHNHSRCVNLPGWYHCECRSGFHDDGTYSLSGESCIDIDECALRTHTCWNDSACINLAGGFDCLCPSGPSCSGDCPHEGGLKHNGQVWILREDRCSVCSCKDGKIFCRRTACDCQNPNVDLFCCPECDTRVTSQCLDQSGQKLYRSGDNWTHSCQQCRCLEGEADCWPLACPSLSCEYTAIFEGECCPRCVSDPCLADNIAYDIRKTCLDSSGISRLSGAVWTMAGSPCTTCQCKNGRVCCSVDLVCLENN

SEQ ID NO: 3 - protein, 810 aa, Rattus norvegicus
MPMDVILVLWFCVCTARTVLGFGMDPDLQLDIISELDLVNTTLGVTQVAGLHNASKAFLFQDVQREIHSAPHVSEKLIQLFRNKSEFTFLATVQQKPSTSGVILSIRELEHSYFELESSGPREEIRYHYIHGGKPRTEALPYRMADGQWHKVALSVSASHLLLHIDCNRIYERVIDPPETNLPPGSNLWLGQRNQKHGFFKGIIQDGKIIFMPNGFITQCPNLNRTCPTCSDFLSLVQGIMDLQELLAKMTAKLNYAETRLGQLENCHCEKTCQVSGLLYRDQDSWVDGDNCGNCTCKSGAVECRRMSCPPLNCSPDSLPVHISGQCCKVCRPKCIYGGKVLAEGQRILTKTCRECRGGVLVKITEACPPLNCSAKDHILPENQCCRVCPGHNFCAEAPKCGENSECKNWNTKATCECKNGYISVQGNSAYCEDIDECAAKMHYCHANTVCVNLPGLYRCDCVPGYIRVDDFSCTEHDDCGSGQHNCDKNAICTNTVQGHSCTCQPGYVGNGTICKAFCEEGCRYGGTCVAPNKCVCPSGFTGSHCEKDIDECAEGFVECHNYSRCVNLPGWYHCECRSGFHDDGTYSLSGESCIDIDECALRTHTCWNDSACINLAGGFDCLCPSGPSCSGDCPHEGGLKHNGQVWILREDRCSVCSCKDGKIFCRRTACDCQNPNVDLFCCPECDTRVTSQCLDQSGQKLYRSGDNWTHSCQQCRCLEGEADCWPLACPSLGCEYTAMFEGECCPRCVSDPCLAGNIAYDIRKTCLDSFGVSRLSGAVWTMAGSPCTTCKCKNGRVCCSVDLECIENN

SEQ ID NO: 4 - protein, 815 aa, Homo sapiens
MESRVLLRTFCLIFGLGAVWGLGVDPSLQIDVLTELELGESTTGVRQVPGLHNGTKAFLFQDTPRSIKASTATAEQFFQKLRNKHEFTILVTLKQTHLNSGVILSIHHLDHRYLELESSGHRNEVRLHYRSGSHRPHTEVFPYILADDKWHKLSLAISASHLILHIDCNKIYERVVEKPTDLPLGTTFWLGQRNNAHGYFKGIMQDVQLLVMPQGFIAQCPDLNRTCPTCNDFHGLVQKIMELQDILAKTSAKLSRAEQRMNRLDQCYCERTCTMKGTTYREFESWIDGCKNCTCLNGTIQCETLICPNPDCPLKSALAYVDGKCCKECKSICQFQGRTYFEGERNTVYSSSGVCVLYECKDQTMKLVESSGCPALDCPESHQITLSHSCCKVCKGYDFCSERHNCMENSICRNLNDRAVCSCRDGFRALREDNAYCEDIDECAEGRHYCRENTMCVNTPGSFMCICKTGYIRIDDYSCTEHDECITNQHNCDENALCFNTVGGHNCVCKPGYTGNGTTCKAFCKDGCRNGGACIAANVCACPQGFTGPSCETDIDECSDGFVQCDSRANCINLPGWYHCECRDGYHDNGMFSPSGESCEDIDECGTGRHSCANDTICFNLDGGYDCRCPHGKNCTGDCIHDGKVKHNGQIWVLENDRCSVCSCQNGFVMCRRMVCDCENPTVDLFCCPECDPRLSSQCLHQNGETLYNSGDTWVQNCQQCRCLQGEVDCWPLPCPDVECEFSILPENECCPRCVTDPCQADTIRNDITKTCLDEMNVVRFTGSSWIKHGTECTLCQCKNGHICCSVDPQCLQEL

SEQ ID NO: 5 - protein, 815 aa, Mus musculus
MESRVLLRTFCVILGLGAVWGLGVDPSLQIDVLTELELGESTDGVRQVPGLHNGTKAFLFQESPRSIKASTATAERFLQKLRNKHEFTILVTLKQIHLNSGVILSIHHLDHRYLELESSGHRNEIRLHYRSGTHRPHTEVFPYILADAKWHKLSLAFSASHLILHIDCNKIYERVVEMPFTDLALGTTFWLGQRNNAHGYFKGIMQDVHVLVMPQGFIAQCPDLNRTCPTCNDFHGLVQKIMELQDILSKTSAKLSRAEQRMNRLDQCYCERTCTVKGTTYRESESWTDGCKNCTCLNGTIQCETLVCPAPDCPPKSAPAYVDGKCCKECKSTCQFQGRSYFEGERNTAYSSSGMCVLYECKDQTMKLVENIGCPPLDCPESHQIALSHSCCKVCKGYDFCSEKHTCMESVCRNLNDRVVCSCRDGFRALREDNAYCEDIDECAEGRHYCRENTMCVNTPGSFMCICKTGYIRIDDYSCTEHDECLTTQHNCDENALCFNTVGGHNCVCKPGYTGNGTTCKAFCKDGCRNGGACIAANVCACPQGFTGPSCETDIDECSEGFVQCDSRANCINLPGWYHCECRDGYHDNGMFAPGGESCEDIDECGTGRHSCTNDTICFNLDGGYDCRCPHGKNCTGDCVHEGKVKHTGQIWVLENDRCSVCSWQTGFVMCRRMVCDCENPTDDLSCCPECDPRLSSQCLHQNGETVYNSGDTWVQDCRQCRCLQGEVDCWPLACPEVECEFSVLPENECCPRCVTDPCQADTIRNDITKTCLDEMNVVRFTGSSWIKHGTECTLCQCKNGHLCCSVDPQCLQEL

SEQ ID NO: 6 - protein, 816 aa, Rattus norvegicus
MESRVLLRTFCVILGLEAVWGLGVDPSLQIDVLSELELGESTAGVRQVPGLHNGTKAFLFQDSPRSIKAPIATAERFFQKLRNKHEFTILVTLKQIHLNSGVILSIHHLDHRYLELESSGHRNEIRLHYRSGTHRPHTEVFPYILADAKWHKLSLAFSASHLILHIDCNKIYERVVEMPSTDLPLGTTFWLGQRNNAHGYFKGIMQDVQLLVMPQGFIAQCPDLNRTCPTCNDFHGLVQKIMELQDILSKTSAKLSRAEQRMNRLDQCYCERTCTMKGATYREFESWTDGCKNCTCLNGTIQCETLVCPAPDCPAKSAPAYVDGKCCKECKSTCQFQGRSYFEGERSTVFSASGMCVLYECKDQTMKLVENAGCPALDCPESHQIALSHSCCKVCKGYDFCSEKHTCMENSVCRNLNDRAVCSCRDGFRALREDNAYCEDIDECAEGRHYCRENTMCVNTPGSFLCICQTGYIRIDDYSCTEHDECLTNQHNCDENALCFNTVGGHNCVCKPGYTGNGTTCKAFCKDGCKNGGACIAANVCACPQGFTGPSCETDIDECSEGFVQCDSRANCINLPGWYHCECRDGYHDNGMFAPGGESCEDIDECGTGRHSCANDTICFNLDGGYDCRCPHGKNCTGDCVHDGKVKHNGQIWVLENDRCSVCSCQTGFVMCQRMVCDCENPTVDLSCCPECDPRLSSQCLHQNGETVYNSGDTWAQDCRQCRCLQEEVDCWPLACPEVECEFSVLPENECCPRCVTDPCQADTIRNDITKTCLDEMNVVRFTGSSWIKHGTECTLCQCKNGHVCCSVDPQCLQEL

SEQ ID NO: 7 - protein, 814 aa, Xenopus laevis
MEFILGIFCVLFCLRAGAGFGVDPSLQIDIFEDLQLGEATPGVQQVQGFHNRSKAFLFQDTSRSIKASTENAERIFQKLRNKHEFTILVTLKQAMLNSGVILSIHHSDHRYLELESSGHRNEVRLHYRSGSHRSQTEVFPYILADDKWHRFSIAISASHLVLHIDCNKIYERIVEKTFMDVPPGTALWVGQRNNVHGYFKGIMQDLQIVVMPQGFISQCPDLNRTCPTCNDFHGLVQKIMELQDILAKTSAKLSRAEQRMNRLDQCYCERSCTVKGNIYRELESWMDGCKKCTCTNGTAQCETLTCSAPNCLSGFSPAYVPGKCCKECQTVCVFQGQMYFEEEREAVYSSSGQCVLFQCKDNTMRRIESPECLPLNCPQSQHITLRNSCCKVCKGHDFCSEGHNCMGYSICKNLDDKAVCICRDGFRALREDNAYCEDIDECTEGRHYCRENTVCVNTPGSFMCVCQTGYLKIDDYSCTEHNECATNQHSCDENAMCFNTVGGHNCVCQPGYTGNGTDCRAFCKDGCRNGGTCIAPNICACPQGFTGPSCESDIDECTEGFVQCDSRANCINLPGWYHCECRDGYHDNGMFSLGGESCEDIDECATGRHSCSNDTVCFNLDGGFDCRCPHGKNCSGDCTHEGKIKHNGQIWVLENDRCSVCSCQVGLVMCRRMVCDCENPTVDLFCCPECDPRLSSQCLHQSGELTYKSGDTWVQNCQQCRCLQGEVDCWPLPCPAIDCEFSVVPESECCPRCVSDPCQADIIRNDITKTCVDETNVVRFTGSSWIKHGTECTLCQCKNGHMCCSVDPQCLQEL

SEQ ID NO: 8 - protein, 816 aa, Gallus gallus
MESGCGLGTLCLLLCLGPVVGFGVDPSLQIDVLSELGLPGYAAGVRQVPGLHNGSKAFLFPDTSRSVKASPETAEIFFQKLRNKYEFTILVTLKQAHLNSGVIFSIHHLDHRYLELESSGHRNEIRLHYRTGSHRSHTEVFPYILADDKWHRLSLAISASHLILHVDCNKIYERVVEKPFMDLPVGTTFWLGQRNNAHGYFKGIMQDVQLLVMPQGFISQCPDLNRTCPTCNDFHGLVQKIMELQDILAKTSAKLSQAEQRMNKLDQCYCERTCTMKGMTYREFESWTDGCKNCTCMNGTVQCEALICSLSDCPPNSALSYVDGKCCKECQSVCIFEGRTYFEGQRETVYSSSGDCVLFECKDHKMQRIPKDSCATLNCPESQQIPLSHSCCKICKGHDFCTEGHNCMEHSVCRNLDDRAVCSCRDGFRALREDNAYCEDVDECAEGQHYCRENTMCVNTPGSFMCICKTGYIRIDDYSCTEHDECVTNQHNCDENALCFNTVGGHNCVCKLGYTGNGTVCKAFCKDGCRNGGACIASNVCACPQGFTGPSCETDIDECSDGFVQCDSRANCINLPGWYHCECRDGYHDNGMFSPSGESCEDIDECATGRHSCANDTVCFNLDGGYDCRCPHGKNCTGDCIHEDKIKHNGQIWVLENDRCSVCSCQSGYVMCRRMVCDCENPTVDLFCCPECDPRLSSQCLHQSGELSYNSGDSWIQNCQQCRCLQGEVDCWPLPCPEVDCEFSVLPENECCPRCVTDPCQADTIRNDITKTCLDETNVVRFTGSSWIKHGTECTLCQCKNGHVCCSVDPQCLQEL

SEQ ID NO: 9 - protein, 237 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRTEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTS

SEQ ID NO: 10 - DNA, 24 bp, Unknown
  NELL1 primer
accttcctgggttatatcgctgtg

SEQ ID NO: 11 - DNA, 20 bp, Unknown
  NELL1 primer
tctcgcagtggcttcctgtg

(19) United States
(12) Patent Application Publication
(10) Pub. No.: US 2010/0167285 A1
(43) Pub. Date: Jul. 1, 2010

(54) METHODS AND AGENTS FOR EVALUATING INFLAMMATORY BOWEL DISEASE, AND TARGETS FOR TREATMENT

(76) Inventors: Stefan Schreiber, Kiel (DE); Philip Rosenstiel, Kiel (DE); Andreas Ruether, Wendtorf (DE); Andre Franke, Kronshagen (DE); John Raelson, Hudson Heights (CA); Randall D. Little, Ste. Dorothee (CA); Tim Keith, Bedford, MA (US); Helene Fournier, Montreal (CA)

Correspondence Address: DOWELL & DOWELL P.C., 103 Oronoco St., Suite 220, Alexandria, VA, 22314, US

(21) Appl. No.: 12/450,478

(22) PCT Filed: Mar. 26, 2008
(86) PCT No.: PCT/US08/58281

§ 371 Date: Mar. 1, 2010

Related U.S. Application Data
(60) Provisional application No. 60/919,953, filed on Mar. 26, 2007.
(60) Provisional application No. 60/907,543, filed on Apr. 6, 2007.

(51) Int. Cl. C12Q 1/68; C07K 2/00; C12N 5/10; C07K 16/00

(52) U.S. Cl. 435/6; 530/300; 536/23.1; 435/366; 530/389.1

(57) ABSTRACT

The invention provides methods for evaluating irritable bowel disease (IBD), including Crohn Disease and Ulcerative Colitis, methods for determining a patient's susceptibility to developing an IBD, and methods for determining a patient's IBD genotype. The invention includes methods, polynucleotides, polypeptides, and antibodies relating to disclosed variants of, and polymorphisms in, the nel-like 1 precursor (NELL1), as well as the 5p13.1 locus, and other genes disclosed herein to be associated with IBD. Thus, the invention provides diagnostic and/or therapeutic targets for IBD, as well as diagnostic and therapeutic agents for IBD.

### PRIORITY

This application claims priority to U.S. Provisional Application Nos. 60/919,953 filed Mar. 26, 2007, and 60/907,543 filed Apr. 6, 2007, each of which is hereby incorporated by reference in its entirety.

### BACKGROUND

An estimated 1.4 million individuals in the United States and 2.2 million individuals in Europe suffer from inflammatory bowel disease [1], a life-long disease that occurs in the form of one of two major sub-phenotypes, Crohn disease (CD) or ulcerative colitis (UC). The pathophysiology of IBD is characterized by a highly activated state of the mucosal immune system and excessive mucosal destruction. The enteric flora appears to play a key role as a stimulating agent [2]. Familial clustering [3,4] and an increased concordance rate of IBD among monozygotic twins [5,6] are hallmarks of the genetic aetiology of IBD, a notion that is further supported by the discovery of several disease genes. These include NOD2 [7-9] (IBD1), a risk haplotype in the 5q31 (IBD5) locus [10,11], DLG5 [12,13], TNFSF15 [14], ATG16L1 [15], CARD4 [16], and IL23R [17].

Yamazaki and colleagues reported the first genome-wide association scan (GWS) for CD, which resulted in the identification of associated polymorphisms in the TNFSF15 gene [14]. Two other GWSs reported the novel CD susceptibility loci IL23R [17] and 5p13.1 [18]. A genome-wide candidate gene analysis was performed using 19,779 non-synonymous SNPs, which led to the identification of a common variant (T300A) in the ATG16L1 gene as predisposing to CD [15], a finding that was later replicated by four other groups [18-21].

Although studies have identified some CD-associated genetic variants, these susceptibility loci explain only a fraction of the heritability of the disease. Thus, identification of additional risk loci for IBD are needed to provide effective diagnostic, prognostic, and therapeutic targets for IBD, including Crohn Disease and ulcerative colitis.

### SUMMARY OF THE INVENTION

Disclosed are risk loci for IBD, which were identified through a multi-stage genome-wide association scan in 393 German cases and 399 German population-representative controls, using the Affymetrix GeneChip® Human Mapping 100K Set [22]. In order to enrich the samples with risk alleles [23] and to reduce phenotypic heterogeneity, CD patients in the GWS were selected for a “severe” phenotype, including a positive IBD family history, age of onset ≦25 years, and no change in diagnosis over the prior five years. The SNPs representing the top 200 association leads were re-genotyped in both a large independent German case control sample and a family-based sample comprising 375 nuclear families. In addition to replicating NOD2, IBD5, and 5p13.1, a novel susceptibility locus was identified on chromosome 11p15.1, namely the nel-like 1 precursor-encoding gene (NELL1).

Thus, in one aspect, the present invention provides a method for evaluating irritable bowel disease (IBD) in a patient suspected of having an IBD, including Crohn Disease. This aspect further relates to a method of determining a patient's susceptibility to developing an IBD, and to methods for determining a patient's IBD genotype. In accordance with some embodiments, the method comprises determining the presence or absence, in a patient's biological sample, of at least one mutation associated with IBD in each of at least two genes listed in Table 1. In accordance with some embodiments, the method involves determining the presence or absence of at least one single nucleotide polymorphism (SNP) in a biological sample from the patient, including at least one the SNP listed in Tables 1-5.

In a related aspect for evaluating IBD, the invention involves determining the presence or absence of at least one mutation in the 11p15.1 locus that is associated with IBD, including mutations in the gene encoding the nel-like 1 precursor (NELL1), in a biological sample from the patient. Such mutations include SNPs localized to the NELL1 gene as disclosed in Tables 1, 2, and 4, as well as mutations encoding the variants R136S, A153T, and/or R354W of the NELL1 polypeptide.

In another related aspect for evaluating IBD, the invention involves determining the presence or absence in a patient's biological sample of: at least one mutation in the 5p13.1 locus that is associated with IBD, including PTGER4 (upstream), and including SNPs disclosed in Tables 1, 3, and 5; and/or at least one mutation associated with IBD in ITGB6 (upstream); and/or at least one mutation associated with IBD in GRM8 (downstream); and/or at least one mutation associated with IBD in OR5V1 (downstream), at least one mutation associated with IBD in PPP3R2 (downstream); and/or at least one mutation associated with IBD in NM_{—}152575 (upstream); and/or at least one mutation associated with IBD in HNF4G (intron).

In a second aspect, the invention provides novel variants of the NELL1 protein and encoding polynucleotides, vectors, and host cells. This aspect further provides antibodies recognizing, in a specific fashion, the novel NELL1 variants. Such products have use as diagnostic, prognostic, and therapeutic targets, and use as diagnostic, prognostic, and therapeutic agents for IBD, including Crohn Disease.

In a third aspect, the invention provides a kit or array containing nucleic acid primers and/or probes for determining the presence and/or absence of an IBD risk genotype in a patient sample. The set of probes and/or primers may consist essentially of primers and/or probes related to evaluating an IBD genotype, and primers and/or probes related to necessary or meaningful assay controls. The kit for evaluating an IBD genotype may comprise nucleic acid probes and/or primers designed to detect ten or more SNPs associated with IBD, such as associated SNPs found in the genes listed in Table 1, and including the SNPs listed in Tables 1-5. Alternatively, the kit for evaluating an IBD genotype may contain probes and/or primers for detecting at least one mutation in NELL1, and optionally at least one mutation associated with an IBD in one or more of NOD2, the 5q31 locus, DLG5, TNFSF15, ATG16L1, CARD4, and IL23R. In accordance with this aspect, the kit may be a companion diagnostic kit for evaluating IBD or determining an IBD genotype in a patient, and for selecting or predicting appropriate therapeutic intervention.

### DESCRIPTION OF THE DRAWINGS

FIG. 1. Expression and localization of NELL1. (A) Transcript levels of NELL1 in a set of different tissues were quantified by RT-PCR. Parallel amplification of β-actin (ACTB) is shown. Expression and localization of the NELL1 protein in healthy colonic tissue is demonstrated in sections (B) (20x) and (C) (40x; bar=10 μm) by immunohistochemistry. Immunoreactivity is confined to mononuclear/lymphocytic cells in the lamina propria (brown DAB reaction product, arrows). A control without the primary antibody is shown in section (D). No major expression differences between colonic specimen from normal controls (N) and Crohn disease (CD) were detected in the Western blot (E) with the same antibody as applied in sections B and C. The single detected band (90 kDa) corresponds to the predicted molecular weight of the isoform encoded by GenBank AK127805 (UniProt accession number Q92832).

FIG. 2. (A) Structure-based multiple sequence alignment of the N-terminal domains of NELL1 and NELL2 homologs (SEQ ID NOS: 1-8) and the N-terminal domain of human thrombospondin-1 (TSP-1) (SEQ ID NO: 9). The DSSP secondary structure assignment of the TSPN structure (PDB code 1z78, chain A) is depicted at the top of the alignment. (B) Multiple sequence alignment of NELL1 and NELL2 homologs (SEQ ID NOS: 1-8). Domain locations are represented at the top of the alignment (VWC domain; EGF-like domain). Alignment columns with more than 70% physicochemically similar amino acids are highlighted. Text labels point to the N-terminal signal peptide and the sequence variants. Residue numbering in the alignment is based on complete protein sequences as derived from corresponding UniProt entries.

FIG. 3. In silico protein analysis. Domain architectures of NELL1/NELL2 and TSP1. The positions of variant amino acids are annotated. Abbreviations are as follows: SP: signal peptide; TSPN: thrombospondin N-terminal domain; CC: coiled-coil region; VWC: von Willebrand factor, type C domain; EGF: EGF domain; TSP-3: thrombospondin-3 repeat; TSP_C: thrombospondin C-terminal domain.

FIG. 4. In silico protein analysis. Computationally derived 3D structure model of the N-terminal domain of the NELL1 protein. The model was created using the TSPN (PDB code 1z78, chain A) as a structure template for NELL1. The locations of variant amino acids as well as of two cysteines forming a disulfide bridge are annotated.

### DESCRIPTION OF THE TABLES

Table 1: Top 200 CD-associated SNPs, ranked with respect to p-values obtained in an allele-(pCCA) or genotype-based (pCCG) case-control comparison in panel A. Also included are pCCA, pCCG, and the transmission disequilibrium test results (pTDT) for the replication panel B. Nucleotide positions refer to NCBI build 34. Markers with p≦0.05 in either the case-control analysis or the transmission disequilibrium test (TDT) in replication panel B are highlighted in bold italics. SNPs with a significant result in both panel B tests are additionally marked by grey shading.

Table 2: Fine mapping of the CD association signal at the NELL1 locus in replication panel B. The p-values of the allele-based (pCCA) and genotype-based (pCCG) association analyses of the tagging SNPs are shown, pTDT is the p-value for the transmission disequilibrium test (TDT). Lead SNPs from the initial screening (see Table 1) are highlighted by grey shading, nonsynonymous SNPs are highlighted. Polymorphisms that are significant in either the TDT or the case-control analyses, are highlighted in bold italics. Pairwise LD is listed using the metric r2 as calculated with Haploview39 and minor allele frequencies (MAF) are listed for control individuals.

Table 3: Fine mapping of the CD association signal at the 5p13.1 locus in replication panel B. The highlighting and the column headers are the same as described in Table 2.

Table 4: Summary of the fine mapping of the NELL1 gene locus in replication panel D. In addition to the p-value for the TDT (pTDT), the corresponding transmitted/untransmitted ratio (T:U) is listed. Other column headers plus the highlighting are the same as described in Table 2.

Table 5: 5p13.1 fine mapping in replication panel D. For a description of the column headers and the highlighting see Table 3.

### DETAILED DESCRIPTION OF THE INVENTION

Crohn disease (CD), a sub-entity of inflammatory bowel disease (IBD), is a complex polygenic disorder. Although recent studies have successfully identified CD-associated genetic variants, these susceptibility loci explain only a fraction of the heritability of the disease. Disclosed is a multi-stage genome-wide scan of 393 German CD cases and 399 controls. Among the 116,161 single-nucleotide polymorphisms tested, an association with the known CD susceptibility gene NOD2, the 5q31 haplotype, and the recently reported CD locus at 5p13.1 were confirmed. In addition, SNP rs1793004 in the gene encoding nel-like 1 precursor (NELL1, chromosome 11p15.1) showed a consistent disease-association in independent German population- and family-based samples (942 cases, 1082 controls, 375 trios). Subsequent fine mapping and replication in an independent sample of 454 French/Canadian CD trios supported the authenticity of the NELL1 association. Further confirmation in a large German ulcerative colitis (UC) sample indicated that NELL1 is a ubiquitous IBD susceptibility locus (combined p<10⁻⁶; OR=1.66, 95% CI: 1.30-2.11). The novel 5p13.1 locus was also replicated in the French/Canadian sample and in an independent UK CD patient panel (453 cases, 521 controls, combined p<10⁻⁶ for SNP rs1992660). Several associations were replicated in at least one independent sample, point to an involvement of ITGB6 (upstream), GRM8 (downstream), OR5V1 (downstream), PPP3R2 (downstream), NM_{—}152575 (upstream) and HNF4G (intron).

Methods for Evaluating IBD and Determining a Patient's IBD Genotype

In one aspect, the present invention provides a method for evaluating irritable bowel disease (IBD) in a patient suspected of having an IBD, including Crohn Disease and ulcerative colitis. This aspect further relates to a method of determining a patient's susceptibility to developing an IBD, and to methods for determining a patient's IBD genotype. In accordance with some embodiments, the method comprises determining the presence or absence in a patient's biological sample, of at least one mutation associated with IBD in each of at least two genes listed in Table 1. In accordance with some embodiments, the method involves determining the presence or absence of at least one single nucleotide polymorphism (SNP) listed in Tables 1-5 in a biological sample from the patient. The SNPs listed in Tables 1-5 are publicly available, and the corresponding nucleotide sequences are hereby incorporated by reference.

In certain embodiments, the method involves determining the presence or absence of two or more SNPs selected from rs2076756 (#1 in Table 1), rs1992662 (#70 in Table 1), rs1992660 (#75 in Table 1), rs1793004 (#83 in Table 1), rs10521209 (#159 in Table 1), and rs2631372 (#163 in Table 1) in the patient sample. Such SNPs localize to the IBD-associated genes and loci of NOD2, 5q31, 5p13.1, and NELL1.

With respect to SNP rs951199 in NELL1 (listed in Table 2), this SNP is further associated with sarciodosis. Thus, the present invention, as it relates to SNP rs951199, further provides a method of diagnosing sarcoidosis in a subject suspected of having the disorder or condition.

In a related aspect, the invention provides a method for evaluating irritable bowel disease (IBD) in a patient suspected of having an IBD, including a method of determining a patient's susceptibility to developing an IBD, and a method for determining an IBD genotype, by using the NELL1 gene as a diagnostic or prognostic target. According to these embodiments, the invention comprises determining the presence or absence of a mutation in the gene encoding the nel-like 1 precursor (NELL1) in a biological sample from the patient, or a mutation in the corresponding 11p15.1 locus. The mutation(s) may include one or more SNPs listed in Tables 1, 2, and 4 that localize to the NELL1 gene, and which are associated with IBD, including: the SNPs rs1793004 and rs951199, and/or the SNPs rs8176785, rs8176786, rs10500885, rs1158547, and rs1945404, and/or may include mutations encoding the Q82R, R136S, A153T, and/or R354W variants of NELL1.

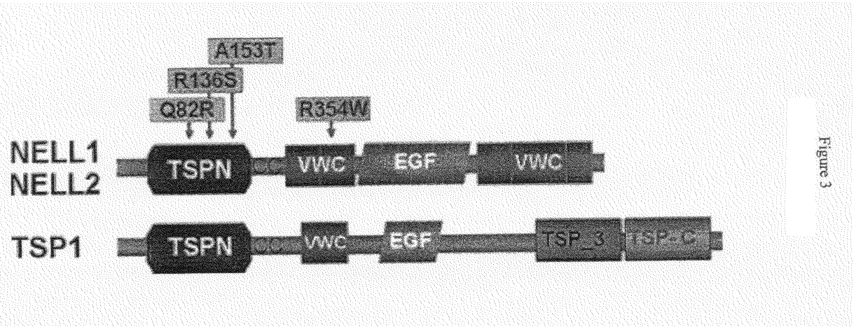

The neural epidermal growth-factor-like (nel) gene was first detected in neural tissue from an embryonic chicken cDNA library, and its human orthologue NELL1 was later discovered in B-cells [48-50]. The arrangement of the functional domains of the 810 aa protein bears resemblance to thrombospondin-1 (TSP-1) and consists of a thrombospondin N-terminal domain (TSPN) and several von Willebrand factor, type C (VWC), and epidermal growth-factor (EGF) domains [51]. As NELL1 binds to, and is phosphorylated by, PKC-β1 via the EGF domains [52], it has been suggested that this protein belongs to a novel class of cell-signalling ligand molecules critical for growth and development. Re-sequencing and fine mapping revealed several non-synonymous SNPs of which the known Q82R variant and the novel R136S and A153T variants affect the TSPN domain, while R354W is located in a VWC domain (FIG. 3) [51]. A153T is close to two highly conserved C-terminal cysteines forming a disulfide bond in the TSPN domain structure of TSP-1 [53] and may cause local conformational changes due to its buried position in the molecule. Generally, the TSPN domain has been shown to serve as a protein-protein interaction module, which binds membrane proteins and proteoglycans and exhibits versatile cell-specific effects on adhesion, migration, and proliferation [54,55]. Since VWC domains occur in numerous proteins of diverse functions and are generally assumed to be involved in protein oligomerization [56], R354W may interfere with NELL1 trimerization [51].

Bone development is severely disturbed in transgenic mice, where over-expression of NELL1 leads to craniosynostis [57] and NELL1 deficiency manifests in skeletal defects due to reduced chondro- and osteogenesis [58]. Interestingly, osteopenia and osteoporosis are leading co-morbidities in IBD patients, even without the use of glucocorticoids [59-61].

In another related aspect, the invention provides a method for evaluating irritable bowel disease (IBD) in a patient suspected of having an IBD, including a method of determining a patient's susceptibility to developing an IBD, and a method for determining an IBD genotype, by using additional loci as diagnostic or prognostic targets. According to these embodiments, the invention comprises determining the presence or absence of a mutation associated with IBD, in a patient's biological sample, in one or more of: the 5p13.1 locus (including associated SNPs of the 5p13.1 locus listed in Tables 1, 3, and 5); PTGER4 (upstream); ITGB6 (upstream); GRM8 (downstream); OR5V1 (downstream); PPP3R2 (downstream); NM_{—}152575 (upstream); and/or HNF4G (intron). In these embodiments, the method may comprise determining the presence or absence of one or more SNPs selected from rs1992662, rs1992660, rs1553575, rs7725523, rs2925757, rs6947579, rs10487428, rs10484545, rs4743484, rs7868736, rs830772, rs272867 in a biological sample (see Table 1).

In certain embodiments, the method further comprises determining the presence or absence of one or more of the following: a mutation in the CARD15 gene associated with an IBD, a mutation in the DLG5 gene associated with an IBD, a mutation in the TNFSF15 gene associated with an IBD, a mutation in the IL23R gene associated with an IBD, and/or a T300A mutation in the ATG16L1 gene. Such genes have previously been shown to be associated with an IBD, and thus, these embodiments may provide diagnostic and prognostic value in combination with those disclosed above (e.g., genes and SNPs listed in Tables 1-5). In accordance with these embodiments, the method may comprise determining the presence and/or absence of the following SNPs in a biological sample: rs2066844 (NOD2), rs2066845 (NOD2), rs2066847 (NOD2), rs1248696 (DLG5), rs2289310 (DLG5), rs11209026 (IL23R), and rs2241880 (ATG16L1).

In accordance with the methods of evaluating IBD, the patient suspected of having an IBD may be female, or may be male, and may be of any age. In some embodiments, however, the patient has a “severe” phenotype, marked in part by onset of the disease before the age of 25, optionally with symptoms of IBD beginning at least three or at least five years prior to testing. Preferably, the patient has a family history of IBD, and/or is already suffering from symptoms of an IBD including Crohn disease or ulcerative colitis at the time of testing. Thus, the methods of the invention, in addition to determining a likelihood of developing an IBD, aid in confirming a diagnosis of an IBD, and provide a means for determining a particular IBD genotype. Knowledge of the particular IBD genotype of the patient will aid in evaluating the likely or potential disease progression as well as the selection of an appropriate therapeutic intervention.

For determining the presence or absence of mutations and/or SNPs in accordance with the invention, samples may be obtained from any part(s) of the patient's body including, but not limited to, hair, mouth, rectum, colon, scalp, blood, dermis, epidermis, and skin cells.

In accordance with these aspects, the presence and/or absence of SNPs or mutations may be determined using a variety of available detection means, including nucleic acid hybridization and/or nucleic acid polymerization assays. For example, in some embodiments, the presence or absence of the mutations and/or SNPs are determined using a gene chip array, a TaqMan assay, or genomic DNA sequencing. The methodology may employ nucleic acid probes and/or primers designed for detecting an SNP or mutation described herein by any available assay format. Detection methods include, but are not limited to, Northern blot analysis, RNase protection, in situ methods, e.g. in situ hybridization, in vitro amplification methods (PCR, LCR, QRNA replicase or RNA-transcription/amplification (TAS, 3SR), reverse dot blot, and other detection assays that are known to those skilled in the art. Further, products obtained by in vitro amplification can be detected according to established methods, e.g. by separating the products on agarose or polyacrylamide gels and by subsequent staining with ethidium bromide or any other dye or reagent. Amplified products may be detected by using labeled primers or labeled dNTPs for amplification. The nucleic acid probes or primers may also be detectably labeled, for example, with a radioisotope, a bioluminescent compound, a chemiluminescent compound, a fluorescent compound, a metal chelate, or an enzyme.

In addition to determining the presence or absence of mutational events in patient nucleic acids, the present invention further provides methods for evaluating IBD by determining the presence or absence of a protein variant, or the level of expression or activity of NELL1 in a biological sample from the patient. The biological sample is preferably from a tissue affected by the IBD, such as the colon, but may optionally be from any tissue expressing NELL1. When determining the presence or absence of a NELL1 variant in a biological sample, the invention involves contacting the sample, or material derived from the sample, with an antibody specific for a NELL1 variant that is associated with IBD, and observing or measuring an antibody-binding event. Antibodies specific for NELL1 variants are described more fully below. Alternatively, when determining the level of expression of NELL1, any antibody recognizing NELL1 may be used, that is, such embodiments are not limited to the use of antibodies against NELL1 variants. Various antibody-based assays for measuring binding between the antibody and NELL1 variant are well known in the art.

NELL1 Polypeptides, Polynucleotide, and Antibodies

In a second aspect, the invention provides novel variants of the NELL1 protein and encoding polynucleotides, as well as antibodies recognizing the novel NELL1 variants. Such products have use as diagnostic, prognostic, and therapeutic targets, as well as diagnostic, prognostic, and therapeutic agents for IBD, including CD.

In accordance with this aspect, the present invention provides novel variants of the NELL1 protein and fragments thereof containing an amino acid mutation disclosed herein. The variants may include one or more of the mutations R136S, A153T, and/or R354W with respect to a mammalian (e.g., human, mouse, or rat) wild-type NELL-1 sequence, or a substantially homologous sequence containing the one or more mutations. Wild-type NELL1 sequences are defined by SEQ ID NOS: 1-3. In certain embodiments, the variant polypeptide further includes the mutation Q82R. These mutations are each associated with the presence of an IBD, and as such, the variant polypeptides find use as diagnostic and therapeutic targets, in conjunction with methods disclosed herein.

This aspect further provides polynucleotides encoding the novel NELL1 variants of the invention, and complementary sequences. Such polynucleotides may be cloned into any suitable vector for replication of the polynucleotides, or expression of the variant polypeptides from promoter sequences, in host cells.

The polynucleotides of the invention may be DNA, cDNA, synthetic DNA, synthetic RNA, or derivatives thereof. Such sequences may be isolated from genomic DNA (e.g., from a patient's cells), and therefore may optionally include naturally occurring introns, genic regions, nongenic regions, and regulatory regions. Alternatively, the polynucleotide may be isolated mRNA, or cDNA produced by reverse transcription, for example. In one embodiment, DNA containing all or part of the coding sequence for a NELL1 variant of the invention, is incorporated into a vector for expression of the encoded polypeptide in suitable host cells. For example, the NELL1 variant encoding sequence may be operably linked to a promoter sequence to drive expression of the NELL1 encoding RNA in a suitable host cell, including a bacterial host (e.g., E. coli), or eukaryotic host cell (e.g., yeast).

The invention may employ any expression vector known in the art, including expression vectors derived from retroviruses, adenovirus, herpes or vaccinia viruses or from various bacterial plasmids. Expression vectors may further be eukaryotic expression vectors, sufficient for delivery of the polynucleotide to organs, tissues or cell populations. Such techniques are well known in the art.

The present invention further provides antibodies that recognize the variant polypeptide sequences of the invention, preferably in a specific fashion over the wild-type sequence. In some embodiments, the antibodies are produced against the variant polypeptide in an animal conventionally used for antibody production, or via an antibody library, which are well known in the art. Such antibodies may recognize a variant NELL1 protein of the invention 2-fold, five-fold, ten-fold, 100-fold, or more, better than the wild-type sequence. Such antibodies are useful as diagnostic and therapeutic agents for IBD, including Crohn Disease.

Antibodies may be prepared by immunizing suitable mammalian hosts utilizing appropriate immunization protocols using the variant proteins of the invention or antigen-containing fragments thereof. To enhance immunogenicity, these proteins or fragments can be conjugated to suitable carriers. Methods for preparing immunogenic conjugates with carriers such as BSA, KLH or other carrier proteins are well known in the art. In some circumstances, direct conjugation using, for example, carbodiimide reagents may be effective; in other instances linking reagents such as those supplied by Pierce Chemical Co. (Rockford, Ill.) may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation. While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, see e.g., Kohler & Milstein (1992) or modifications which affect immortalization of lymphocytes or spleen cells, as is generally known. The immortalized cell lines secreting the desired antibodies can be screened by immunoassay in which the antigen is the peptide hapten, polypeptide or protein. When the appropriate immortalized cell culture secreting the desired antibody is identified, the cells can be cultured either in vitro or by production in ascites fluid. The desired monoclonal antibodies may be recovered from the culture supernatant or from the ascites supernatant. Fragments of the monoclonal antibodies or the polyclonal antisera which contain the immunologically significant portion(s) can be used as antagonists, as well as the intact antibodies. Use of immunologically reactive fragments, such as Fab or Fab′ fragments, is often preferable, especially in a therapeutic context, as these fragments are generally less immunogenic than the whole immunoglobulin. The antibodies or fragments may also be produced, using current technology, by recombinant means. Antibody regions that bind specifically to the desired regions of the protein can also be produced in the context of chimeras derived from multiple species. Antibody regions that bind specifically to the desired regions of the protein can also be produced in the context of chimeras from multiple species, for instance, humanized antibodies. The antibody can therefore be a humanized antibody or a human antibody, as described in U.S. Pat. No. 5,585,089 or Riechmann et al. (1988).

Diagnostic Kits

The invention further provides a kit or array containing nucleic acid primers and/or probes for determining the presence and/or absence of IBD risk genotype in a patient sample. The kit may consist essentially of primers and/or probes related to evaluating an IBD genotype in a sample, and primers and/or probes related to necessary or meaningful assay controls. The kit for evaluating IBD may comprise nucleic acid probes and/or primers designed to detect ten or more SNPs associated with IBD, such as SNPs found in the genes listed in Table 1, and including the SNPs listed in Tables 1-5. Alternatively, the kit for evaluating IBD (e.g., for evaluating or determining an IBD genotype) may contain probes and/or primers for detecting at least one mutation in NELL1 that is associated with IBD, or at least one mutation in the 5p13.1 locus that is associated with IBD, and optionally at least one mutation associated with an IBD in one or more of NOD2, the 5q31 locus, DLG5, TNFSF15, ATG16L1, CARD4, and IL23R (including SNPs described herein). In accordance with this aspect, the kit may be a companion diagnostic kit for evaluating IBD or determining an IBD genotype in a patient, and for selecting or predicting appropriate therapeutic intervention.

In certain embodiments, the kit comprises probes and/or primers designed to detect the presence or absence of two or more SNPs selected from rs2076756 (#1 in Table 1), rs1992662 (#70 in Table 1), rs1992660 (#75 in Table 1), rs1793004 (#83 in Table 1), rs10521209 (#159 in Table 1), and rs2631372 (#163 in Table 1) in the patient sample. Such SNPs localize to the IBD-associated genes and loci of NOD2, 5q31, 5p13.1, and NELL1.

The kits may comprise, or may further comprise, probes and/or primers designed to detect the presence or absence of a mutation in the NELL1 gene in a biological sample, or a mutation in the corresponding 11p15.1 locus. The mutation(s) may include one or more SNPs listed in Table 1, 2, and 4 that localize to the NELL1 gene, and which are associated with IBD, including the SNPs rs1793004 and rs951199, and/or may include rs8176785, rs10500885, rs1158547, and rs1945404, and/or may include mutations encoding the Q82R, R136S, A153T, and/or R354W variants of NELL1.

The kits may also comprise, or further comprise, probes and/or primers designed to detect the presence or absence of a mutation associated with IBD, in a patient's biological sample, in one or more of: the 5p13.1 locus (including SNPs listed in Tables 1, 3, and 5), including PTGER4 (upstream); ITGB6 (upstream); GRM8 (downstream); OR5V1 (downstream), PPP3R2 (downstream); NM_{—}152575 (upstream); and HNF4G (intron). In these embodiments, the set of probes and/or primers may comprise probes and/or primers designed to detect one or more SNPs selected from rs1992662, rs1992660, rs1553575, rs7725523, rs2925757, rs6947579, rs10487428, rs10484545, rs4743484, rs7868736, and rs830772 in a biological sample.

The kit may include a set of probes and/or primers designed to detect at least 10 IBD-associated polymorphisms (e.g., comprising or consisting essentially of IBD-associated genes disclosed herein), or may be designed to detect 20, 50, 100, 200, or more IBD-associated polymorphisms.

In accordance with this aspect, the probes and primers may comprise antisense nucleic acids or oligonucleotides that are wholly or partially complementary to the diagnostic targets described herein. The probes and primers will be designed to detect the particular diagnostic target via an available nucleic acid detection assay format, which are well known in the art.

In this context, the term “oligonucleotide” refers to naturally-occurring species or synthetic species formed from naturally-occurring subunits or their close homologs. The term may also refer to moieties that function similarly to oligonucleotides, but have non-naturally-occurring portions. Thus, oligonucleotides may have altered sugar moieties or inter-sugar linkages. Exemplary among these are phosphorothioate and other sulfur containing species which are known in the art. Such substitutions may comprise phosphorothioate bonds, methyl phosphonate bonds, or short chain alkyl or cycloalkyl structures. Oligonucleotides may also include species that include at least some modified base forms. Thus, purines and pyrimidines other than those normally found in nature may be so employed. Similarly, modifications on the furanosyl portions of the nucleotide subunits may also be effected. Examples of such modifications are 2′-O-alkyl- and 2′-halogen-substituted nucleotides. Some non-limiting examples of modifications at the 2′ position of sugar moieties which may be useful include OH, SH, SCH₃, F, OCH₃, OCN, O(CH₂), NH₂ and O(CH₂)n CH₃, where n is from 1 to about 10. Such oligonucleotides are functionally interchangeable with natural oligonucleotides or synthesized oligonucleotides, which have one or more differences from the natural structure. All such analogs are comprehended by this invention so long as they function effectively to hybridize with at least one diagnostic target of the invention.

The oligonucleotides in accordance with this invention may comprise from about 3 to about 50 subunits or nucleotides. In some embodiments, oligonucleotides and analogs comprise from about 8 to about 25 subunits (e.g., nucleotides) and still more preferred to have from about 12 to about 20 subunits or nucleotides. As defined herein, a “subunit” is a base and sugar combination suitably bound to adjacent subunits through phosphodiester or other bonds.

The kits of the invention may comprise probes and/or primers designed to detect the diagnostic targets via detection methods that include amplification, restriction enzyme cleavage, hybridization, sequencing, and cleavage.

Amplification methods include: self sustained sequence replication (Guatelli et al., 1990), transcriptional amplification system (Kwoh et al., 1989), Q-Beta Replicase (Lizardi et al., 1988), isothermal amplification (e.g. Dean et al., 2002; and Hafner et al., 2001), or any other nucleic acid amplification method, followed by the detection of the amplified molecules using techniques well known to those of ordinary skill in the art. These detection schemes are especially useful for the detection of nucleic acid molecules if such molecules are present in very low number.

Restriction enzyme cleavage methods include: isolating sample and control DNA, amplification (optional), digestion with one or more restriction endonucleases, determination of fragment length sizes by gel electrophoresis and comparing samples and controls. Differences in fragment length sizes between sample and control DNA indicates mutations in the sample DNA. Moreover, sequence specific ribozymes (see, e.g., U.S. Pat. No. 5,498,531 or DNAzyme e.g. U.S. Pat. No. 5,807,718) can be used to score for the presence of specific mutations by development or loss of a ribozyme or DNAzyme cleavage site.

Hybridization methods include any measurement of the hybridization or gene expression levels, of sample nucleic acids to probes, and include microarray technology to detect several (e.g., more than 10, more than 100, or more than 1000) diagnostic targets. Thus, SNPs and mutations of the invention can be detected in a sample by hybridizing sample nucleic acids, e.g., DNA or RNA, to high density arrays or bead arrays containing oligonucleotide probes designed to hybridize thereto. Methods of forming high density arrays of oligonucleotides with a minimal number of synthetic steps are known. The oligonucleotide analogue array can be synthesized on a single or on multiple solid substrates by a variety of methods, including, but not limited to, light-directed chemical coupling, and mechanically directed coupling.

Nucleic acid hybridization simply involves contacting a probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization tolerates fewer mismatches. One of skill in the art will appreciate that hybridization conditions may be selected to provide any degree of stringency.

In a preferred embodiment, hybridization is performed at low stringency to ensure hybridization and then subsequent washes are performed at higher stringency to eliminate mismatched hybrid duplexes. Successive washes may be performed at increasingly higher stringency until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Probes based on the sequences of the genes described above may be prepared by any commonly available method. Oligonucleotide probes for screening or assaying a tissue or cell sample are preferably of sufficient length to specifically hybridize only to appropriate, complementary genes or transcripts. Typically the oligonucleotide probes will be at least about 10, 12, 14, 16, 18, 20 or 25 nucleotides in length. In some cases, longer probes of at least 30, 40, or 50 nucleotides will be desirable.

The phrase “hybridizing specifically to” or “specifically hybridizes” refers to the binding, duplexing, or hybridizing of a molecule substantially to or only to a particular nucleotide sequence or sequences under stringent conditions when that sequence is present in a complex mixture (e.g., total cellular) DNA or RNA.

As used herein a “probe” is defined as a nucleic acid, capable of binding to a target nucleic acid of complementary sequence through one or more types of chemical bonds, usually through complementary base pairing, usually through hydrogen bond formation. As used herein, a probe may include natural (i.e., A, G, U, C, or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

A variety of sequencing reactions known in the art can be used to directly sequence nucleic acids for the presence or the absence of one or more polymorphisms or mutations (such as those described herein). Examples of sequencing reactions include those based on techniques developed by Maxam and Gilbert (1977) or Sanger (1977). It is also contemplated that any of a variety of automated sequencing procedures can be utilized, including sequencing by mass spectrometry (see, e.g. PCT International Publication No. WO 94/16101; Cohen et al., 1996; and Griffin et al., 1993), real-time pyrophosphate sequencing method (Ronaghi et al., 1998; and Permutt et al., 2001) and sequencing by hybridization (see e.g. Drmanac et al., 2002).

Other methods of detecting polymorphisms include methods in which protection from cleavage agents is used to detect mismatched bases in RNA/RNA, DNA/DNA or RNA/DNA heteroduplexes (Myers et al., 1985). In general, the technique of “mismatch cleavage” starts by providing heteroduplexes formed by hybridizing (labeled) RNA or DNA containing a wild-type sequence with potentially mutant RNA or DNA obtained from a sample. The double-stranded duplexes are treated with an agent who cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of a mutation or SNP (see, for example, Cotton et al., 1988; and Saleeba et al., 1992). In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping polymorphisms. For example, the mutY enzyme of E. coli cleaves A at G/A mismatches (Hsu et al., 1994). Other examples include, but are not limited to, the MutHLS enzyme complex of E. coli (Smith and Modrich Proc. 1996) and Cel 1 from the celery (Kulinski et al., 2000) both cleave the DNA at various mismatches. According to an exemplary embodiment, a probe based on a polymorphic site is hybridized to a cDNA or other DNA product from a test cell or cells. The duplex is treated with a DNA mismatch repair enzyme, and the cleavage products, if any, can be detected from electrophoresis protocols or the like. See, for example, U.S. Pat. No. 5,459,039. Alternatively, the screen can be performed in vivo following the insertion of the heteroduplexes in an appropriate vector. The whole procedure is known to those ordinary skilled in the art and is referred to as mismatch repair detection (see e.g. Fakhrai-Rad et al., 2004).

In other embodiments, alterations in electrophoretic mobility can be used to identify polymorphisms in a sample. For example, single strand conformation polymorphism (SSCP) analysis can be used to detect differences in electrophoretic mobility between mutant and wild type nucleic acids (Orita et al., 1989; Cotton et al., 1993; and Hayashi 1992). Single-stranded DNA fragments of case and control nucleic acids will be denatured and allowed to renature. The secondary structure of single-stranded nucleic acids varies according to sequence. The resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the method utilizes heteroduplex analysis to separate double stranded heteroduplex molecules on the basis of changes in electrophoretic mobility (Kee et al., 1991).

In yet another embodiment, the movement of mutant or wild-type fragments in a polyacrylamide gel containing a gradient of denaturant is assayed using denaturing gradient gel electrophoresis (DGGE) (Myers et al., 1985). When DGGE is used as the method of analysis, DNA will be modified to insure that it does not completely denature, for example by adding a GC clamp of approximately 40 by of high-melting GC-rich DNA by PCR. In a further embodiment, a temperature gradient is used in place of a denaturing gradient to identify differences in the mobility of control and sample DNA (Rosenbaum et al., 1987). In another embodiment, the mutant fragment is detected using denaturing HPLC (see e.g. Hoogendoorn et al., 2000).

Examples of other techniques for detecting polymorphisms include, but are not limited to, selective oligonucleotide hybridization, selective amplification, selective primer extension, selective ligation, single-base extension, selective termination of extension or invasive cleavage assay. For example, oligonucleotide primers may be prepared in which the polymorphism is placed centrally and then hybridized to target DNA under conditions which permit hybridization only if a perfect match is found (Saiki et al., 1986; Saiki et al., 1989). Such oligonucleotides are hybridized to PCR amplified target DNA or a number of different mutations when the oligonucleotides are attached to the hybridizing membrane and hybridized with labeled target DNA. Alternatively, the amplification, the allele-specific hybridization and the detection can be done in a single assay following the principle of the 5′ nuclease assay (e.g. see Livak et al., 1995). For example, the associated allele, a particular allele of a polymorphic locus, or the like is amplified by PCR in the presence of both allele-specific oligonucleotides, each specific for one or the other allele. Each probe has a different fluorescent dye at the 5′ end and a quencher at the 3′ end. During PCR, if one or the other or both allele-specific oligonucleotides are hybridized to the template, the Taq polymerase via its 5′ exonuclease activity will release the corresponding dyes. The latter will thus reveal the genotype of the amplified product.

Hybridization assays may also be carried out with a temperature gradient following the principle of dynamic allele-specific hybridization or like e.g. Jobs et al., (2003); and Bourgeois and Labuda, (2004). For example, the hybridization is done using one of the two allele-specific oligonucleotides labeled with a fluorescent dye, and an intercalating quencher under a gradually increasing temperature. At low temperature, the probe is hybridized to both the mismatched and full-matched template. The probe melts at a lower temperature when hybridized to the template with a mismatch. The release of the probe is captured by an emission of the fluorescent dye, away from the quencher. The probe melts at a higher temperature when hybridized to the template with no mismatch. The temperature-dependent fluorescence signals therefore indicate the absence or presence of an associated allele, a particular allele of a polymorphic locus, or the like (e.g. Jobs et al., 2003). Alternatively, the hybridization is done under a gradually decreasing temperature. In this case, both allele-specific oligonucleotides are hybridized to the template competitively. At high temperature none of the two probes are hybridized. Once the optimal temperature of the full-matched probe is reached, it hybridizes and leaves no target for the mismatched probe (e.g. Bourgeois and Labuda, 2004). In the latter case, if the allele-specific probes are differently labeled, then they are hybridized to a single PCR-amplified target. If the probes are labeled with the same dye, then the probe cocktail is hybridized twice to identical templates with only one labeled probe, different in the two cocktails, in the presence of the unlabeled competitive probe.

Alternatively, allele specific amplification technology that depends on selective PCR amplification may be used in conjunction with the present invention. Oligonucleotides used as primers for specific amplification may carry the associated allele, a particular allele of a polymorphic locus, or the like, also referred to as “mutation” of interest in the center of the molecule, so that amplification depends on differential hybridization (Gibbs et al., 1989) or at the extreme 3′ end of one primer where, under appropriate conditions, mismatch can prevent, or reduce polymerase extension (Prossner, 1993). In addition it may be desirable to introduce a novel restriction site in the region of the mutation to create cleavage-based detection (Gasparini et al., 1992). It is anticipated that in certain embodiments, amplification may also be performed using Taq ligase for amplification (Barany, 1991). In such cases, ligation will occur only if there is a perfect match at the 3′ end of the 5′ sequence making it possible to detect the presence of a known associated allele, a particular allele of a polymorphic locus, or the like at a specific site by looking for the presence or absence of amplification. The products of such an oligonucleotide ligation assay can also be detected by means of gel electrophoresis. Furthermore, the oligonucleotides may contain universal tags used in PCR amplification and zip code tags that are different for each allele. The zip code tags are used to isolate a specific, labeled oligonucleotide that may contain a mobility modifier (e.g. Grossman et al., 1994).

In yet another alternative, allele-specific elongation followed by ligation will form a template for PCR amplification. In such cases, elongation will occur only if there is a perfect match at the 3′ end of the allele-specific oligonucleotide using a DNA polymerase. This reaction is performed directly on the genomic DNA and the extension/ligation products are amplified by PCR. To this end, the oligonucleotides contain universal tags allowing amplification at a high multiplex level and a zip code for SNP identification. The PCR tags are designed in such a way that the two alleles of a SNP are amplified by different forward primers, each having a different dye. The zip code tags are the same for both alleles of a given SNPs and they are used for hybridization of the PCR-amplified products to oligonucleotides bound to a solid support, chip, bead array or like. For an example of the procedure, see Fan et al. (Cold Spring Harbor Symposia on Quantitative Biology, Vol. LXVIII, pp. 69-78 2003).

Another alternative includes the single-base extension/ligation assay using a molecular inversion probe, consisting of a single, long oligonucleotide (see e.g. Hardenbol et al., 2003). In such an embodiment, the oligonucleotide hybridizes on both side of the SNP locus directly on the genomic DNA, leaving a one-base gap at the SNP locus. The gap-filling, one-base extension/ligation is performed in four tubes, each having a different dNTP. Following this reaction, the oligonucleotide is circularized whereas unreactive, linear oligonucleotides are degraded using an exonuclease such as exonuclease I of E. coli. The circular oligonucleotides are then linearized and the products are amplified and labeled using universal tags on the oligonucleotides. The original oligonucleotide also contains a SNP-specific zip code allowing hybridization to oligonucleotides bound to a solid support, chip, and bead array or like. This reaction can be performed at a high multiplexed level.

In another alternative, the associated allele, a particular allele of a polymorphic locus, or the like is scored by single-base extension (see e.g. U.S. Pat. No. 5,888,819). The template is first amplified by PCR. The extension oligonucleotide is then hybridized next to the SNP locus and the extension reaction is performed using a thermostable polymerase such as ThermoSequenase (GE Healthcare) in the presence of labeled ddNTPs. This reaction can therefore be cycled several times. The identity of the labeled ddNTP incorporated will reveal the genotype at the SNP locus. The labeled products can be detected by means of gel electrophoresis, fluorescence polarization (e.g. Chen et al., 1999) or by hybridization to oligonucleotides bound to a solid support, chip, and bead array or like. In the latter case, the extension oligonucleotide will contain a SNP-specific zip code tag.

In yet another alternative, a SNP is scored by selective termination of extension. The template is first amplified by PCR and the extension oligonucleotide hybridizes in the vicinity of the SNP locus, close to but not necessarily adjacent to it. The extension reaction is carried out using a thermostable polymerase such as ThermoSequenase (GE Healthcare) in the presence of a mix of dNTPs and at least one ddNTP. The latter has to terminate the extension at one of the allele of the interrogated SNP, but not both such that the two alleles will generate extension products of different sizes. The extension product can then be detected by means of gel electrophoresis, in which case the extension products need to be labeled, or by mass spectrometry (see e.g. Storm et al., 2003).

In another alternative, SNPs are detected using an invasive cleavage assay (see U.S. Pat. No. 6,090,543). There are five oligonucleotides per SNP to interrogate but these are used in a two step-reaction. During the primary reaction, three of the designed oligonucleotides are first hybridized directly to the genomic DNA. One of them is locus-specific and hybridizes up to the SNP locus (the pairing of the 3′ base at the SNP locus is not necessary). There are two allele-specific oligonucleotides that hybridize in tandem to the locus-specific probe but also contain a 5′ flap that is specific for each allele of the SNP. Depending upon hybridization of the allele-specific oligonucleotides at the base of the SNP locus, this creates a structure that is recognized by a cleavase enzyme (U.S. Pat. No. 6,090,606) and the allele-specific flap is released. During the secondary reaction, the flap fragments hybridize to a specific cassette to recreate the same structure as above except that the cleavage will release a small DNA fragment labeled with a fluorescent dye that can be detected using regular fluorescence detector. In the cassette, the emission of the dye is inhibited by a quencher.

Methods of Treatment

The present invention provides a method for treating IBD, including ulcerative colitis and Crohn Disease, by administering an effective amount of a NELL1 polypeptide or functional portion thereof, to a patient in need. Administration of the NELL1 polypeptide to patients suffering from IBD, or at risk of developing an IBD, may be effective to mitigate the effects of absent, partial inactivation, or abnormal expression of endogenous NELL1.

The present invention provides methods of treating an IBD by expressing in vivo a NELL1 polynucleotide. These nucleic acids can be inserted into any of a number of well-known vectors for the transfection of target cells and organisms as described below. The nucleic acids are transfected into cells, ex vivo or in vivo, through the interaction of the vector and the target cell. The nucleic acids encoding a NELL1 protein or NELL1 sequence, under the control of a promoter, express the encoded protein, thereby mitigating the effects of absent, partial inactivation, or abnormal expression of endogenous NELL1.

Alternatively, the invention provides nucleic acids, including expression constructs, that, when introduced into host cells expressing NELL1, express antisense and dsRNAs corresponding to portions of the NELL1 gene. Such expression of antisense and dsRNAs is effective to silence endogenous NELL1 expression via antisense or RNAi-mediated gene silencing.

Such gene therapy procedures have been used to correct acquired and inherited genetic defects, cancer, and viral infection in a number of contexts. The ability to express artificial genes in humans facilitates the prevention and/or cure of many important human disorders, including many disorders which are not amenable to treatment by other therapies (for a review of gene therapy procedures, see Anderson, 1992; Nebel & Feigner, 1993; Mitani & Caskey, 1993; Mulligan, 1993; Dillon, 1993; Miller, 1992; Van Brunt, 1998; Vigne, 1995; Kremer & Perricaudet 1995; Doerfler & Bohm 1995; and Yu et al., 1994).

Delivery of the gene or genetic material into the cell is the first critical step in gene therapy treatment of a disorder. A large number of delivery methods are well known to those of skill in the art. Preferably, the nucleic acids are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see the references included in the above section.

The use of RNA or DNA based viral systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., 1992; Johann et al., 1992; Sommerfelt et al., 1990; Wilson et al., 1989; Miller et al., 1999; and PCT/US94/05700).

In applications where transient expression of the nucleic acid is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., 1987; U.S. Pat. No. 4,797,368; WO 93/24641; Kotin, 1994; Muzyczka, 1994). Construction of recombinant AAV vectors is described in a number of publications, including U.S. Pat. No. 5,173,414; Tratschin et al., 1985; Tratschin, et al., 1984; Hermonat & Muzyczka, 1984; and Samulski et al., 1989.

In particular, numerous viral vector approaches are currently available for gene transfer in clinical trials, with retroviral vectors by far the most frequently used system. All of these viral vectors utilize approaches that involve complementation of defective vectors by genes inserted into helper cell lines to generate the transducing agent. pLASN and MFG-S are examples are retroviral vectors that have been used in clinical trials (Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors (Ellem et al., 1997; and Dranoff et al., 1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 by inverted terminal repeats flanking the transgene expression cassette. Efficient gene transfer and stable transgene delivery due to integration into the genomes of the transduced cell are key features for this vector system (Wagner et al., 1998, Kearns et al., 1996).

Replication-deficient recombinant adenoviral vectors (Ad) are predominantly used in transient expression gene therapy; because they can be produced at high titer and they readily infect a number of different cell types. Most adenovirus vectors are engineered such that a transgene replaces the Ad E1a, E1b, and E3 genes; subsequently the replication defector vector is propagated in human 293 cells that supply the deleted gene function in trans. Ad vectors can transduce multiple types of tissues in vivo, including nondividing, differentiated cells such as those found in the liver, kidney and muscle tissues. Conventional Ad vectors have a large carrying capacity. An example of the use of an Ad vector in a clinical trial involved polynucleotide therapy for antitumor immunization with intramuscular injection (Sterman et al., 1998). Additional examples of the use of adenovirus vectors for gene transfer in clinical trials include Rosenecker et al., 1996; Sterman et al., 1998; Welsh et al., 1995; Alvarez et al., 1997; Topf et al., 1998.

Packaging cells are used to form virus particles that are capable of infecting a host cell. Such cells include 293 cells, which package adenovirus, and PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., 1995, reported that Moloney murine leukemia virus can be modified to express human heregulin fused to gp70, and the recombinant virus infects certain human breast cancer cells expressing human epidermal growth factor receptor. This principle can be extended to other pairs of viruses expressing a ligand fusion protein and target cells expressing a receptor. For example, filamentous phage can be engineered to display antibody fragments (e.g., Fab or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application. Alternatively, vectors can be delivered to cells ex vivo, such as cells explanted from an individual patient (e.g., lymphocytes, bone marrow aspirates, and tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA), and re-infused back into the subject organism (e.g., patient). Various cell types suitable for ex vivo transfection are well known to those of skill in the art (see, e.g., Freshney et al., 1994; and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow.

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and lad (differentiated antigen presenting cells).

Vectors (e.g., retroviruses, adenoviruses, liposomes, etc.) containing therapeutic nucleic acids can be also administered directly to the organism for transduction of cells in vivo. Alternatively, naked DNA can be administered.

Administration is by any of the routes normally used for introducing a molecule into ultimate contact with blood or tissue cells, as described above. The nucleic acids are administered in any suitable manner, preferably with the pharmaceutically acceptable carriers described above. Suitable methods of administering such nucleic acids are available and well known to those of skill in the art, and, although more than one route can be used to administer a particular composition, a particular route can often provide a more immediate and more effective reaction than another route (see Samulski et al., 1989). The present invention is not limited to any method of administering such nucleic acids, but preferentially uses the methods described herein.

In other embodiments, the invention provides methods of treating IBD, by administering antibodies, including synthetic antibodies and antibody fragments, specific for NELL1 to patients in need of treatment. The antibodies are generally administered in amounts effective to inhibit NELL1 receptor or ligand binding. For example, such antibodies may block NELL1 receptor binding in vivo, thereby restoring normal levels NELL1 activity. The antibodies may be directed, for example, to the EGF or TSPN domains of NELL1. Alternatively, mimetics of NELL1 may be prepared, including antidiotypic antibodies, effective to act as agonists at the NELL1 receptor. Various forms of antibodies sufficient for these purposes are described elsewhere herein.

Screening Assays

The invention further provides methods of screening for agonists and antagonists, and compounds or agents that modulate the expression, of NELL1. Such compounds and agents find use in the treatment of IBD, including the development and manufacture of treatments for IBD.

In this aspect, the invention comprises contacting the NELL1 polypeptide, for example, as expressed in a suitable host cell, or as present in a suitable in vitro system, with a test compound or agent. The level of NELL1 activity (as described in the art, and as measured via any suitable NELL1 assay described in the art), or in some cases the level of NELL1 expression, may then be compared to controls to identify an agonist or antagonist of NELL1 (or an up- or down-regulator as the case may be). Controls may include positive controls for NELL1 activation or expression, and appropriate negative controls.

In certain embodiments, the method may employ the NELL1 variants described herein, in order to identify a suitable agonist or antagonist of the NELL1 variant.

Agents that are assayed in the above method can be randomly selected or rationally selected or designed. As used herein, an agent is said to be randomly selected when the agent is chosen randomly without considering the specific sequences involved in the association of the protein of the invention alone or with its associated substrates, binding partners, etc. An example of randomly selected agents is the use of a chemical library or a peptide combinatorial library, or a growth broth of an organism. As used herein, an agent is said to be rationally selected or designed when the agent is chosen on a non-random basis which takes into account the sequence of the target site or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site. The agents of the present invention can be, as examples, oligonucleotides, antisense polynucleotides, interfering RNA, peptides, peptide mimetics, antibodies, antibody fragments, small molecules, vitamin derivatives, as well as carbohydrates. Peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-gene-encoded amino acids are to be included.

Another class of agents useful in this aspect includes antibodies or fragments thereof that bind to NELL1 or a variant thereof. Antibody agents can be obtained by immunization of suitable mammalian subjects with peptides, containing as antigenic regions, those portions of the protein intended to be targeted by the antibodies (see section above of antibodies as probes for standard antibody preparation methodologies).

In yet another class of agents, the present invention includes peptide mimetics that mimic the three-dimensional structure of NELL1 or a variant thereof. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others. In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. In another form, peptide analogs are commonly used in the pharmaceutical industry as non-peptide drugs with properties analogous to those of the template peptide. These types of non-peptide compounds are also referred to as peptide mimetics or peptidomimetics (Fauchere, 1986; Veber & Freidinger, 1985; Evans et al., 1987) which are usually developed with the aid of computerized molecular modeling. Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage using methods known in the art. Labeling of peptide mimetics usually involves covalent attachment of one or more labels, directly or through a spacer (e.g., an amide group), to non-interfering position(s) on the peptide mimetic that are predicted by quantitative structure-activity data and molecular modeling. Such non-interfering positions generally are positions that do not form direct contacts with the macromolecule(s) to which the peptide mimetic binds to produce the therapeutic effect. Derivitization (e.g., labeling) of peptide mimetics should not substantially interfere with the desired biological or pharmacological activity of the peptide mimetic. The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al., 1991) and the phage display method. The two hybrid method detects protein-protein interactions in yeast (Fields et al., 1989). The phage display method detects the interaction between an immobilized protein and a protein that is expressed on the surface of phages such as lambda and M13 (Amberg et al., 1993; Hogrefe et al., 1993). These methods allow positive and negative selection for protein-protein interactions and the identification of the sequences that determine these interactions.

### Examples

### Methods

Patient Recruitment

German patients and controls in panels A, B, and C partially overlap with samples that have been used in other studies before [12,15,65,66]. Panels A and B almost completely overlap with the panels (also termed panel A and B) that were used in a recently published IBD association screen of non-synonymous SNPs [15]. In this non-synonymous SNP scan no coding SNPs that were evaluable were located in NELL1. All patients were recruited at the Charite University Hospital (Berlin, Germany) and the Department of General Internal Medicine of the Christian-Albrechts-University (Kiel, Germany), with the support of the German Crohn and Colitis Foundation. Clinical, radiological and endoscopic (i.e. type and distribution of lesions) examinations were required to unequivocally confirm the diagnosis of Crohn disease or ulcerative colitis [67,68], and histological findings also had to be confirmative of, or compatible with, the diagnosis. In the case of uncertainty, patients were excluded from the study. German control individuals were obtained from the POPGEN biobank [25].

The UK patients (Panel E) were recruited as described [69]; UK controls were obtained from the 1958 British Birth Cohort.

The French/Canadian trios (Panel D) were sampled from the Quebec founder population (QFP). Membership of the founder population was defined as having four grandparents with French Canadian family names who were born in the Province of Quebec, Canada, or in adjacent areas of the Provinces of New Brunswick and Ontario, or in New England or New York State (USA).

Informed written consent was obtained from all study participants. All collection protocols were approved by the institutional review committees of the participating centres.

SNP Genotypinq with the Affymetrix 100k Gene Chip Array

Genotyping of cases and controls was carried out using the Affymetrix GeneChip® Human Mapping 50K Xba and Hind Arrays (Affymetrix, Santa Clara, Calif., USA). Genotypes were called by the GeneChip® DNA Analysis Software (GDAS v2.0, Affymetrix). Gender was verified by counting the heterozygous SNPs on the X chromosome. Quality checks further comprised the verification of individual sample call rates (≧90%) and, to ensure that no samples were confused, the 31 identical SNPs present on both chips were checked for identical genotypes for the same individual. SNPs that had a low genotyping success rate (<90%), were monomorphic, or deviated from Hardy-Weinberg equilibrium (p≦0.01) were eliminated from subsequent analyses. Experimental details concerning the genotyping of the 100k SNP set are provided in Matsuzaki et al. [22].

Follow-Up Genotypinq and Sequencing

SNPlex™ (Applied Biosystems, Foster City, Calif., USA) genotyping of panels A, B, and C was carried out as recently described [15]. Genotype concordance rates for SNPs rs1793004, rs1992660, and rs1992662 were checked using TaqMan (Applied Biosystems) as an independent genotyping technology on an automated platform [70] and the functionally tested assays C______392093_{—}10, C______11472026_{—}10, and C______11472042_{—}10. All three concordance rates were >98%, excluding genotyping errors as a potential source of false-positive associations. The same three TaqMan assays were also used to genotype panels C and E. Genotypes for panel D were generated at Genizon BioSciences using the Illumina GoldenGate™ platform (Illumina, San Diego, Calif., USA). All process data were logged into, and administered by, a database-driven LIMS [71]. TaqMan genotyping of NOD2/Arg702Trp, NOD2/Gly908Arg, NOD2/Leu1007fs, DLG5/Arg30Gln, DLG5/Pro1371Gln, DLG5/e26, and ATG16L1/Thr300Ala was performed using previously described assays [12,15,72]. IL23R/Arg381Gln, NELL1/rs8176785, and NELL1/rs8176786 were genotyped in panels A, B, C, and E using the functionally tested assay C______1272298_{—}10, C______3203197_{—}10, and C______32647553_{—}10, respectively (Applied Biosystems). Prior to statistical analyses, the same cut-off criteria as described above for the 100k analysis (p_{HWE}>0.01, MAF_{controls}>0, callrate≧90%) were applied to the SNPs under study.

Sequencing of genomic DNA was performed using Applied Biosystems BigDye™ chemistry according to the supplier's recommendations. Traces were inspected for the presence of SNPs and InDels using novoSNP [73].

Statistical Analysis

Genome-wide data analysis was carried out using an updated version of GENOMIZER [74]. Association hits that passed the quality criteria were extracted using the “GenomizerHits” tool. Haploview 4.0 [75] was used for association analysis, transmission disequilibrium tests, and LD quantification of the replication data. Fisher's exact test was used when appropriate. The supplementary p-value plots and quantile-quantile plots were created using R. Single-marker disease associations and possible marker-marker interactions were assessed for statistical significance by means of logistic regression analysis (forward selection), as implemented in the procedure LOGISTIC of the SAS software package (SAS Institute, Cary N.C., USA). Haplotype analyses were carried out using COCAPHASE 2.403 [76] and PHASE 2.1 [77,78].

RT-PCR, Western Blot and Immunohistochemistry

For the assessment of tissue-specific expression patterns, a commercial tissue panel was employed (Clontech, Palo Alto, Calif., USA). Primers used for amplification of NELL1 were (NELL1_{—}14-16_F ACCTTCCTGGGTTATATCGCTGTG (SEQ ID NO: 10) and NELL1_{—}14-16_R TCTCGCAGTGGCTTCCTGTG (SEQ ID NO: 11), expected amplicon length: 285 bp). The following conditions were applied: denaturation for 5 min at 95° C.; 40 cycles of 30 sec at 95° C., 20 sec at 60° C., 45 sec at 72° C.; final extension for 10 min at 72° C. To confirm the use of equal amounts of RNA in each experiment, all samples were checked in parallel for β-actin mRNA expression. All amplified DNA fragments were analyzed on 2% agarose gels and subsequently documented by a BioDoc Analyzer (Biometra, Göttingen, Germany).

Paraformaldehyde-fixed paraffin-embedded biopsies from normal controls (n=6) and from patients with confirmed colonic CD (n=6) were analysed. Two slides of each biopsy were stained with hematoxylin-eosin for routine histological evaluation. The other slides were subjected to a citrate-based antigen retrieval procedure, permeabilized by incubation with 0.1% Triton X-100 in 0.1M phosphate-buffered saline (PBS), washed three times in PBS and blocked with 0.75% bovine serum albumin in PBS for 20 minutes. Sections were subsequently incubated with the primary antibody (anti-NELL1, Abnova, mouse monoclonal) at a 1:500 dilution in 0.75% BSA overnight at 4° C. After washing in PBS, tissue-bound antibody was detected using biotinylated goat-anti mouse (Vector Laboratory, Burlingame, Calif.) followed by HRP-conjugated avidin, both diluted at 1:100 in PBS. Controls were included using irrelevant primary antibodies as well as omitting the primary antibodies using only secondary antibodies and/or HRP-conjugated avidin. No significant staining was observed with any of these controls (data not shown). Bound antibody was detected by standard chromogen technique (Vector Laboratory) and visualized by an Axiophot microscope (Zeiss, Jena, Germany). Pictures were captured by a digital camera system (Axiocam, Zeiss).

Western blot analysis was performed as described [79]. In brief, 20 μg of protein lysates freshly derived from colonic biopsies of four healthy controls without any obvious intestinal pathology and four CD patients with confirmed ileal and colonic inflammation were lysed, separated by SDS polyacrylamide gel electrophoresis and transferred to PVDF membrane by standard techniques. NELL1 was detected using the same monoclonal anti-NELL1 antibody also employed for immunohistochemistry.

In Silico Protein Analysis

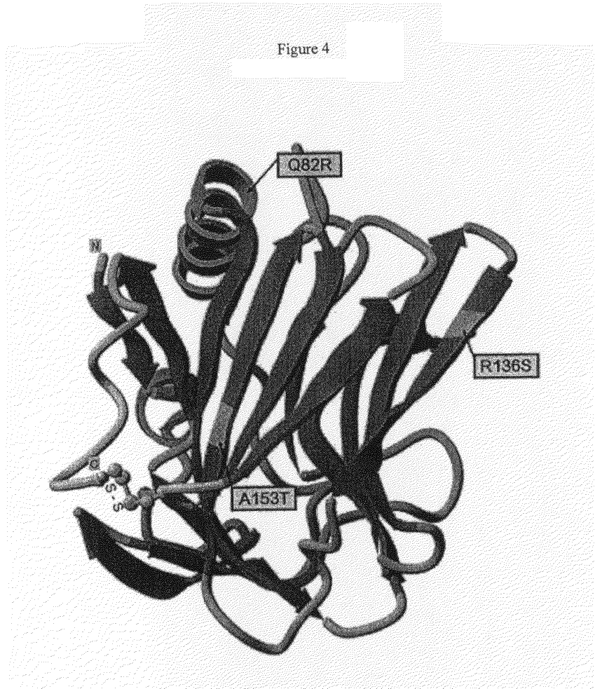

Aligned sequences were retrieved from the UniProt database and protein domain architectures taken from the NCBI conserved domain search website. To predict the 3D structure of the N-terminal domain of NELL1, we explored the fold recognition results returned by the web servers GenTHREADER and FFAS03. Based upon the very similar server predictions, a pair-wise sequence-structure alignment of NELL1 to the crystal structure of the human thrombospondin-1 N-terminal domain (TSPN) was constructed as input for the 3D-modeling server WHATIF, which returned a structure model of the NELL1 N-terminal domain (FIG. 4).

Genome-Wide Association Scan

A total of 116,161 SNPs were genotyped in case-control panel A. Of these, 92,387 SNPs had a call rate ≧90%, were polymorphic in panel A, and showed no significant departure from Hardy-Weinberg equilibrium (p_{HWE}≦0.01 in controls). At an unadjusted per-test significance level of 5%, the experiment had 80% power to detect an odds ratio of 1.6, and 33% power to detect an odds ratio of 1.3, assuming that 20% of the controls were carriers of the risk factor. The GWS results were not corrected for potential population substructure because (i) very low (<10⁻³) F_{ST} values have previously been reported for different geographic regions of Germany [24], (ii) patients of panel A were all selected from the Northern part of Germany, and were therefore geographically matched to the population-representative controls from the POPGEN biobank [25], (iii) quantile-quantile plots, which can help to identify spurious association results [26], revealed no inflation of the X² statistics, and (iv) replication criteria included confirmation by family-based association tests (transmission disequilibrium test, TDT), which are robust against population stratification [27].

Replication

The 200 most significant SNPs in the GWS were next genotyped in two independent German samples. “Replication” was considered to have been achieved if the p-values of both, the case-control analysis and the family-based TDT were <5%. Replication in two independent samples also rendered test-wise Bonferroni correction superfluous, which would have been overly conservative in a replication setting anyway [28]. In addition to rs2631372 (#163), which localizes to the 5q31 haplotype [10], an association with CD was confirmed for rs2076756 (#1, p_{CCA}<10⁻¹²) and rs10521209 (#159) in NOD2 [7-9]. The recently reported 5p13.1 locus [18] was also replicated (rs1992660/#70, rs1992662/#75), and a novel susceptibility gene, NELL1, was identified (rs1793004/#83). While only these six SNPs were found to be nominally significant in both, the TDT and the case-control analysis, and therefore fulfilled the formal replication criteria, 47 SNPs were significant in only one test, including two more SNPs in the 5p13.1 region (#79, #105), one in NELL1 (#116), and one in the IBD5 region (#191). In view of the low power of the TDT, it appears worth mentioning that use of p_{CCA} or p_{CCG}<10⁻² as the sole replication criterion would have led to the additional acceptance of rs2925757 (ITGB6, upstream), rs6947579 (GRM8, downstream), rs10484545 (OR5V1, downstream), rs4743484 (PPP3R2, downstream), rs7868736 (NM_{—}152575, upstream), and rs830772 (HNF4G, intron) as confirmed associations.

We did not detect our previously reported CD associations of ATG16L1 [15], and DLG5 [12] in this screening and did not see any association with and IL23R [16]. However, SNP coverage around these genes was very low. In order to benchmark our experiments, relevant SNPs in these genes were therefore genotyped in panels A and B, using TaqMan technology, and a disease association was observed for SNPs in all three genes. Interestingly, haplotype A-tagging SNP e26 in the DLG5 gene was replicated (over-transmission of common allele T, T:U=275:219, p=0.012), while the associations of non-synonymous SNPs Arg30Gln and Pro1371Gln did not reach statistical significance.

To corroborate our main association findings, we examined the significantly associated NELL1 and 5p13.1 SNPs in two additional, independent Caucasian CD samples: Panel D, which comprised population-based Falk-Rubinstein trios from the Quebec founder population (QFP), and panel E, a case-control sample from the UK. The NELL1 association was replicated in the QFP (over-transmission of the common C allele, T:U=140:107, p=0.036) sample. In addition, the association of 5p13.1 SNP 1992660 was replicated in the QFP case-control sample (p=0.0081) and the combined p-value for panels B, D, and E was 1.24×10⁻⁷ in an allele-based test. The odds ratio for homozygosity of the common A allele was 1.36 (95% CI: 1.36-2.04). In the UK sample (panel E), the associations of SNPs rs1992660 and rs1992662 were replicated with p-values (allelic X² test) of 0.036 and 0.0011, respectively, while the NELL1 SNP association did not achieve formal significance.

Evaluation in Ulcerative Colitis (UC)

The three SNPs rs1793004 (NELL1), rs1992660, and rs1992662 (both 5p13.1) with a confirmed CD association were also analysed in a German UC panel (panel C, 1059 single patients and 419 trios). The NELL1 SNP rs1793004 also showed a disease association in the UC case-control panel (p=0.0017 in the allele-based X² test) and the odds ratio for homozygosity of the common C allele was 1.54 (95% CI: 1.08-2.20). Given the similar odds ratio in UC and CD (1.76; 95% CI: 1.27-2.45), NELL1 appears to be a ubiquitous IBD susceptibility gene (combined p<10⁻⁶; OR=1.66, 95% CI: 1.30-2.11). No association to UC was detected for the 5p13.1 locus.

Fine Mapping Around NELL1

Fine mapping around the NELL1 gene was carried out in replication panels B and D using HapMap tagging SNPs at a density of 8 kb. Twenty-one SNPs in the NELL1 gene yielded a p-value<0.05 in the single-point analyses of panel B (12 in panel D), of which several markers were significant in both, the TDT and case-control test. Results were not corrected for multiple testing because the association between CD and the NELL1 locus was regarded as established through the previous analyses of panels A and B.

NELL1 comprises several regions of increased recombination, scattered over a total of 906 kb. Disease associations were found with various small linkage disequilibrium (LD) blocks, suggesting the existence of more than one causal variant in the gene. In a logistic regression analysis of the combined panels A+B, the best model fit was achieved with SNPs rs1793004, rs951199, rs8176785, rs10500885, rs1158547, and rs1945404. The main association peak was located 5′ of the gene, although a few significant associations were also found towards the 3′ end. The signal sharply declines 5′ of the NELL1 gene, thereby excluding an involvement of the proximate SLC6A5 gene. A gender-stratified analysis (data not shown) of the 117 SNPs in panel B confirmed a disease association in both genders.

Detection of Additional DNA Sequence Variants

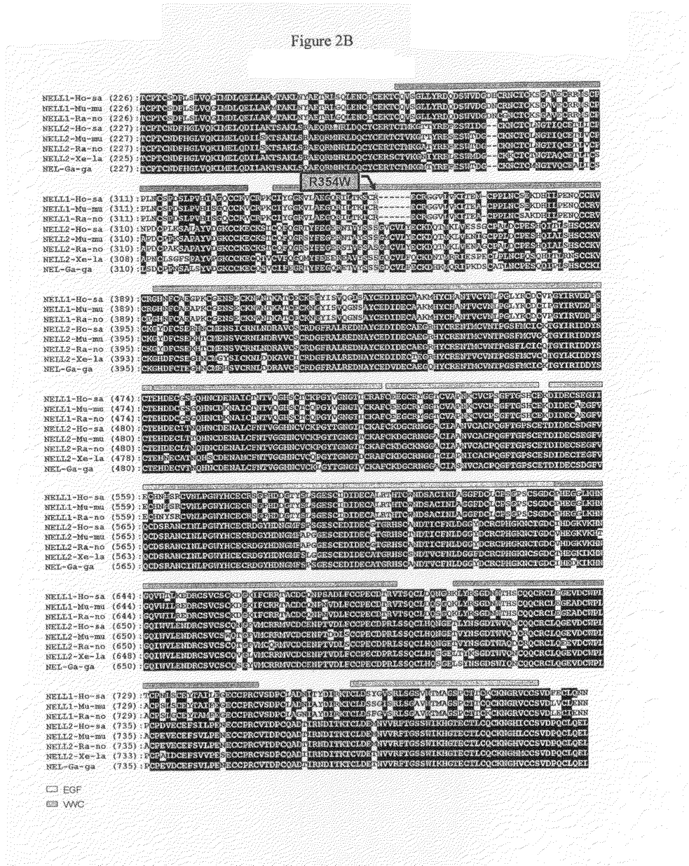

Since rs1793004 clearly localizes to NELL1, a systematic search for additional, potentially disease-associated variants in the gene was carried out by re-sequencing all exons, splice sites, and the promoter region in 47 CD patients. Apart from verifying 26 already annotated variants, five new polymorphisms were identified, two of which represented novel non-synonymous SNPs (nsSNPs): NELL01_{—}02 (R136S) and NELL01_{—}03 (A153T). Both nsSNPs were located in exon 4 and mapped to the thrombospondin N-terminal domain (TSPN) of the NELL1 protein. Two known, common nsSNPs were verified among the 26 annotated SNPs, namely rs8176785 (Q82R) in exon 3 and rs8176786 (R354W) in exon 10. Variant Q82R is located in the TSPN domain, while R354W resides in a von Willebrand factor type C (VWC) domain. In-silico analysis, including multiple sequence alignment of NELL homologues and structural modeling of the TSPN domain, revealed a strong conservation of the variant positions (FIGS. 2 and 3).

The novel nsSNPs were too rare to warrant formal statistical analysis (total occurrence of heterozygotes in panel B (CD/controls): 2/0 for R136S and 10/9 for A153T). While common nsSNP rs8176786 showed a disease association in panel E (p=0.048), the second common nsSNP, rs8176785, was significantly associated with CD in panel B (p=0.039), and with UC in panel C (p=0.013). The combined p-value in the full German IBD sample (A+B+C) was 0.0048 in a genotype-based X² test (2 degrees of freedom).

Expression and Localization of NELL1 within the Intestinal Mucosa

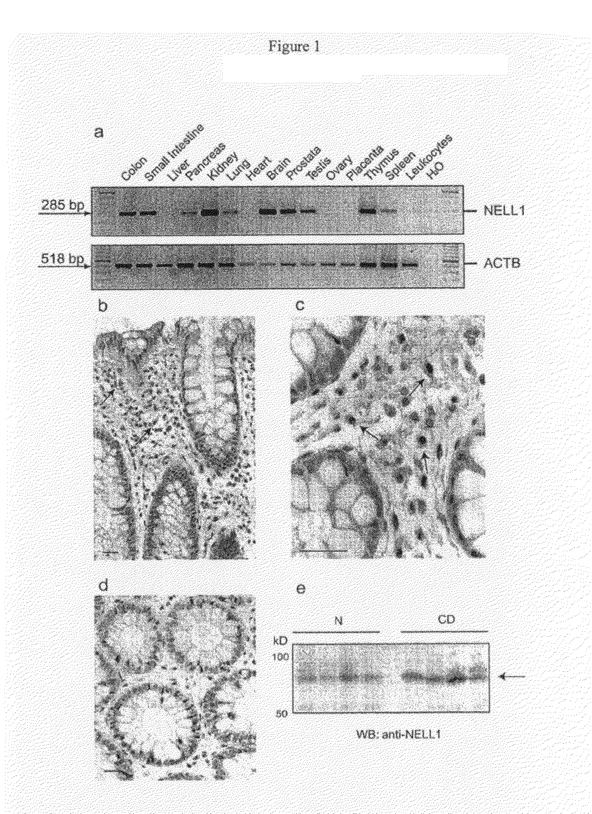

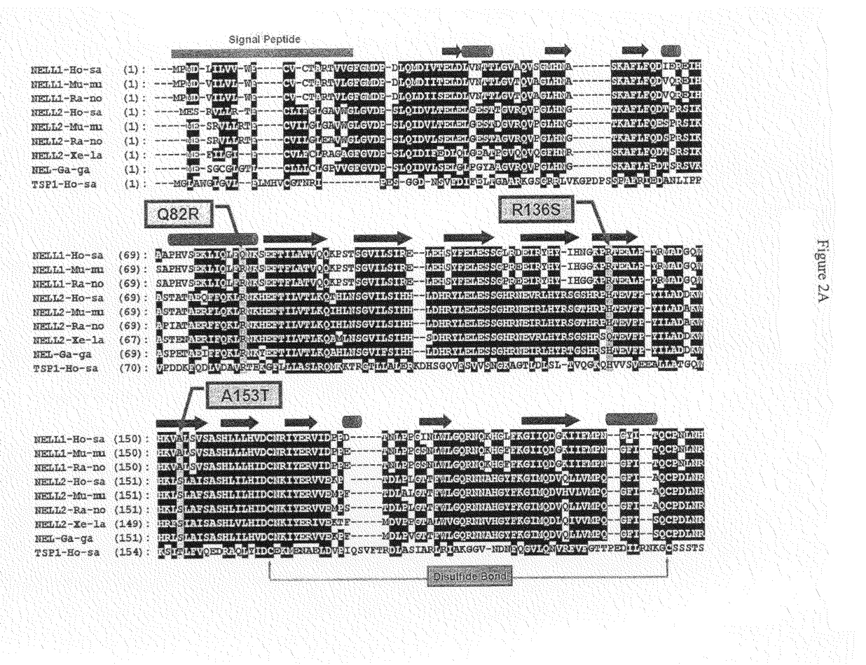

When NELL1 transcript levels were investigated by RT-PCR in a tissue panel, high expression became apparent in small intestine, kidney, prostate, and brain, whereas moderate expression was seen in colonic mucosa and in immune-relevant organs/cells such as thymus and spleen (FIG. 1A). The localization of NELL1 in the colonic mucosa was investigated by immunohistochemistry (FIGS. 1B and 1C). Immunoreactivity was confined to large mononuclear cells in the lamina propria. In Western blot experiments using colonic biopsy specimen from normal controls and CD patients (FIG. 1E), the antibody recognized a single 90 kDa band corresponding to the correct size of the annotated NELL1 transcript (AK127805). Real-time quantitative PCR revealed no significant difference between normal and patient tissue.

Fine Mapping of 5p13.1

The 650 kb susceptibility region on 5p13.1, upstream of the PTGER4 gene, was subjected to fine mapping in panels B (1 SNP/24 kb) and D (1 SNP/3 kb). Several SNPs showed a consistent disease association in both panels. The strongest effect in the combined case-control panel (A+B) was seen for SNP rs1553575 (odds ratio for homozygotes of the common G allele: 1.78; 95% CI: 1.32-2.40).

Interestingly, the gender-stratified analysis of 5p13.1 SNPs showed that the association signal was stronger in females than in males, suggesting that females carrying the predisposing allele(s) of this locus are at higher risk to develop Crohn disease than their male counterparts. To have comparable power, the same number of male and female individuals were randomly drawn from the combined panel (378 controls, 343 cases).

Interaction with Known Disease Loci

Logistic regression analysis and a Breslow-Day test for odds ratio heterogeneity were used to analyse the full German case-control panel (A+B) for potential epistatic effects. No statistically significant interactions were observed, neither between polymorphisms within the NELL1 gene (rs1793004) or the 5p13.1 region (rs1992660 and rs1992662), nor between these loci and any of the known disease-associated variants in IL23R (rs11209026/Arg381Gln), NOD2 (rs2066844/Arg702Trp, rs2066845/Gly908Arg, rs2066847/Leu1007fs), ATG16L1 (rs2241880/T300A), DLG5 (rs1248696/Arg30Gln), or in the IBD5 region (tagging SNP IGR2063_b1 [11,29]).

### DISCUSSION

We have identified NELL1 as a novel disease gene for Crohn disease (CD), a result that was obtained in a genome-wide casecontrol association scan with 116,161 SNPs and by extensive replication in three independent samples from three distinct ethnicities. In a recently published GWAS from the UK population [30,31] (1,748 CD patients and 2,938 controls genotyped), the NELL1 region was covered with 263 SNPs. Of these, 23 SNPs were significantly associated with CD (p<0.05 under an additive or general model) and six SNPs had a p<0.01: rs7122630, rs4475916, rs7115151, rs11025862, rs2063913, rs11026037. The NELL1 region was not subjected to replication in the UK scan, since none of the 23 SNPs fell below the chosen cut-off (p<010⁵).

In addition to identifying NELL1 as a CD risk factor, we also replicated the disease association recently described for the 5p13.1 region [18]. The genome-wide scan also confirmed two of the previously known CD loci, NOD2 and 5q31, but it should be pointed out that the marker set only covered 31% of the genome [32,33]. The previously established disease association of neither IL23R [17], nor DLG5 [12], nor ATG16L1 [15], were detected. However, not a single SNP, for example, in the ATG16L1 gene was present on the Affymetrix GeneChip® Human Mapping 100K Set and coverage of all these genes was low. Targeted post-hoc genotyping of the relevant SNPs in the German screening and replication panel was therefore carried out and confirmed the CD associations of ATG16L1, IL23R and DLG5 in our study sample. We replicated the association of a haplotype A-tagging SNP in DLG5 which is supported by several other replications of the association between DLG5 and CD association [34-42]. Results for the two nonsynonymous SNPs in DLG5 were not reaching significance. We do not consider these SNPs as causative at this point. They are either part of a larger number of putatively causative SNPs not yet discovered or mere additional markers for unknown causative variants. We expect further relevant, hitherto unknown and rare variants in DLG5 that may only be detectable by large-scale sequencing of the gene [43]. It should also be noted that the DLG5 association has not been replicated in each and every population analysed so far [44-46]. Recent studies have proposed gender and/or age at onset-related associations of DLG5 [13,34,40-42] that would require exact matching of the study groups to become detectable. Our sample used in this study contained mainly CD patients with disease onset during early adulthood (average age at onset >22 years). This may have contributed to a replication that was weaker than the original description in statistical terms (for review see [47]).

The targeted replication of the CD association of ATG16L1, IL23R and DLG5 also serves to illustrate the highly conservative criteria employed in our study, which may have resulted in an under-appreciation of most initial association findings. Using these criteria, ATG16L1/Thr300Ala and IL23R/Arg381Gln would not have been included in the follow-up because the p-values of the two variants (0.014 for Thr300Ala and 0.0027 for Arg381Gln) both exceeded the cut-off of 0.0021 (attained by rs3790889 as number 200 of the ranked SNP list). Therefore, future efforts to replicate the major findings of our study should also include those SNPs that yielded a significant p-value in only one of the replication panels.

The neural epidermal growth-factor-like (net) gene was first detected in neural tissue from an embryonic chicken cDNA library, and its human orthologue NELL1 was later discovered in B-cells [48-50]. The arrangement of the functional domains of the 810 aa protein bears resemblance to thrombospondin-1 (TSP-1) and consists of a thrombospondin N-terminal domain (TSPN) and several von Willebrand factor, type C (VWC), and epidermal growth-factor (EGF) domains [51]. As NELL1 binds to, and is phosphorylated by, PKC-β1 via the EGF domains [52], it has been suggested that this protein belongs to a novel class of cell-signalling ligand molecules critical for growth and development. Re-sequencing and fine mapping revealed several non-synonymous SNPs of which the known Q82R variant and the novel R136S and A153T variants affect the TSPN domain, while R354W is located in a VWC domain (FIG. 3) [51]. A153T is close to two highly conserved C-terminal cysteines forming a disulfide bond in the TSPN domain structure of TSP-1 [53] and may cause local conformational changes due to its buried position in the molecule. Generally, the TSPN domain has been shown to serve as a protein-protein interaction module, which binds membrane proteins and proteoglycans and exhibits versatile cell-specific effects on adhesion, migration, and proliferation [54,55]. Since VWC domains occur in numerous proteins of diverse functions and are generally assumed to be involved in protein oligomerization [56], R354W may interfere with NELL1 trimerization [51]

Bone development is severely disturbed in transgenic mice, where over-expression of NELL1 leads to craniosynostis [57] and NELL1 deficiency manifests in skeletal defects due to reduced chondro- and osteogenesis [58]. Interestingly, osteopenia and osteoporosis are leading co-morbidities in IBD patients, even without the use of glucocorticoids [59-61]. PTGER4, which has been suggested as the causative gene in the 5p13.1 locus, is among the key genes that are down-regulated in NELL1-deficient mice [58]. However, no statistical interaction was seen in our study between the NELL1 and 5p13.1 SNPs.

The replication criteria used in our study were particularly strict and required a significant p-value in both, the family-based and the case-control association test in two different populations. Other studies used less stringent criteria for the replication of genome-wide association findings, and based their conclusions upon a single independent case control sample only [14,17]. With such criteria, several additional SNPs would have been considered replicated in our study, some of which point towards genes putatively involved in the pathophysiology of IBD. Integrin beta 6 (ITGB6), for example, regulates activation of TGF-β [62], a cytokine that has been established as an anti-inflammatory regulator in TNF-related CD pathopysiology [63,64]. Two hits point towards the glutamate pathway, namely glutamate receptor type 8 (GRM8) and glutamate receptor, ionotropic, N-methyl-D-aspartate 3A (GRIN3A, formerly PPP3R2). Normal glutamate metabolism has been found to be important for the maintenance of intestinal function. Finally, SNP rs7868736 is located approximately 100 kb upstream of the ZNF618 gene encoding a zinc finger protein clearly expressed in human colon.

In summary, we have successfully performed a systematic genome-wide association scan in Crohn disease that led to the identification of the NELL1 gene on chromosome 11p15.1 as a novel susceptibility gene for IBD. We confirmed 5p13.1 as a CD-associated locus relating to PTGER4 that is probably regulated by NELL1.

### REFERENCES

1. Loftus E V Jr (2004) Clinical epidemiology of inflammatory bowel disease: Incidence, prevalence, and environmental influences. Gastroenterology 126: 1504-1517.
2. Schreiber S, Rosenstiel P, Albrecht M, Hampe J, Krawczak M (2005) Genetics of Crohn disease, an archetypal inflammatory barrier disease. Nat Rev Genet 6: 376-388.
3. Orholm M, Munkholm P, Langholz E, Nielsen O H, Sorensen T I, et al. (1991) Familial occurrence of inflammatory bowel disease. N Engl J Med 324: 84-88.
4. Kuster W, Pascoe L, Purrmann J, Funk S, Majewski F (1989) The genetics of Crohn disease: complex segregation analysis of a family study with 265 patients with Crohn disease and 5,387 relatives. Am J Med Genet 32: 105-108.
5. Tysk C, Lindberg E, Jarnerot G, Floderus-Myrhed B (1988) Ulcerative colitis and Crohn's disease in an unselected population of monozygotic and dizygotic twins. A study of heritability and the influence of smoking. Gut 29: 990-996.
6. Thompson N P, Driscoll R, Pounder R E, Wakefield A J (1996) Genetics versus environment in inflammatory bowel disease: results of a British twin study. Bmj 312: 95-96.
7. Ogura Y, Bonen D K, Inohara N, Nicolae D L, Chen F F, et al. (2001) A frameshift mutation in NOD2 associated with susceptibility to Crohn's disease. Nature 411: 603-606.
8. Hugot J P, Chamaillard M, Zouali H, Lesage S, Cezard J P, et al. (2001) Association of NOD2 leucine-rich repeat variants with susceptibility to Crohn's disease. Nature 411: 599-603.
9. Hampe J, Cuthbert A, Croucher P J, Mirza M M, Mascheretti S, et al. (2001) Association between insertion mutation in NOD2 gene and Crohn's disease in German and British populations. Lancet 357: 1925-1928.
10. Peltekova V D, Wintle R F, Rubin L A, Amos C I, Huang Q, et al. (2004) Functional variants of OCTN cation transporter genes are associated with Crohn disease. Nat Genet 36: 471-475.
11. Rioux J D, Daly M J, Silverberg M S, Lindblad K, Steinhart H, et al. (2001) Genetic variation in the 5q31 cytokine gene cluster confers susceptibility to Crohn disease. Nat Genet 29: 223-228.
12. Stoll M, Corneliussen B, Costello C M, Waetzig G H, Mellgard B, et al. (2004) Genetic variation in DLG5 is associated with inflammatory bowel disease. Nat Genet 36: 476-480.
13. Blank V, Friedrichs F, Babusukumar U, Wang T, Stoll M, et al. (2007) DLG5 R30Q variant is a female-specific protective factor in pediatric onset Crohn's disease. Am J Gastroenterol 102: 391-398.
14. Yamazaki K, McGovern D, Ragoussis J, Paolucci M, Butler H, et al. (2005) Single nucleotide polymorphisms in TNFSF15 confer susceptibility to Crohn's disease. Hum Mol Genet 14: 3499-3506.
15. Hampe J, Franke A, Rosenstiel P, Till A, Teuber M, et al. (2007) A genome-wide association scan of nonsynonymous SNPs identifies a susceptibility variant for Crohn disease in ATG16L1. Nat Genet 39: 207-211.
16. McGovern D P, Hysi P, Ahmad T, van Heel D A, Moffatt M F, et al. (2005) Association between a complex insertion/deletion polymorphism in NOD1 (CARD4) and susceptibility to inflammatory bowel disease. Hum Mol Genet 14: 1245-1250.
17. Duerr R H, Taylor K D, Brant S R, Rioux J D, Silverberg M S, et al. (2006) A genome-wide association study identifies IL23R as an inflammatory bowel disease gene. Science 314: 1461-1463.
18. Libioulle C, Louis E, Hansoul S, Sandor C, Farnir F, et al. (2007) Novel Crohn Disease Locus Identified by Genome-Wide Association Maps to a Gene Desert on 5p13.1 and Modulates Expression of PTGER4. PLoS Genet. 3: e58.
19. Cummings J R, Cooney R, Pathan S, Anderson C A, Barrett J C, et al. (2007) Confirmation of the role of ATG16I1 as a Crohn's disease susceptibility gene. Inflamm Bowel Dis.
20. Rioux J D, Xavier R J, Taylor K D, Silverberg M S, Goyette P, et al. (2007) Genome-wide association study identifies new susceptibility loci for Crohn disease and implicates autophagy in disease pathogenesis. Nat Genet 39: 596-604.
21. Prescott N J, Fisher S A, Franke A, Hampe J, Onnie C M, et al. (2007) A Nonsynonymous SNP in ATG16L1 Predisposes to Ileal Crohn's Disease and Is Independent of CARD15 and IBD5. Gastroenterology 132: 1665-1671.
22. Matsuzaki H, Dong S, Loi H, Di X, Liu G, et al. (2004) Genotyping over 100,000 SNPs on a pair of oligonucleotide arrays. Nat Methods 1: 109-111.
23. Fingerlin T E, Boehnke M, Abecasis G R (2004) Increasing the power and efficiency of disease-marker case-control association studies through use of allele sharing information. Am J Hum Genet 74: 432-443.
24. Steffens M, Lamina C, Illig T, Bettecken T, Vogler R, et al. (2006) SNP-based analysis of genetic substructure in the German population. Hum Hered 62: 20-29.
25. Krawczak M, Nikolaus S, von Eberstein H, Croucher P J, El Mokhtari N E, et al. (2006) PopGen: population-based recruitment of patients and controls for the analysis of complex genotype-phenotype relationships. Community Genet 9: 55-61.
26. Clayton D G, Walker N M, Smyth D J, Pask R, Cooper J D, et al. (2005) Population structure, differential bias and genomic control in a large-scale, casecontrol association study. Nat Genet 37: 1243-1246.
27. Spielman R S, McGinnis R E, Ewens W J (1993) Transmission test for linkage disequilibrium: the insulin gene region and insulin-dependent diabetes mellitus (IDDM). Am J Hum Genet 52: 506-516.
28. McIntyre L M, Martin E R, Simonsen K L, Kaplan N L (2000) Circumventing multiple testing: a multilocus Monte Carlo approach to testing for association. Genet Epidemiol 19: 18-29.
29. Fisher S A, Hampe J, Onnie C M, Daly M J, Curley C, et al. (2006) Direct or indirect association in a complex disease: the role of SLC22A4 and SLC22A5 functional variants in Crohn disease. Hum Mutat 27: 778-785.
30. Parkes M, Barrett J C, Prescott N J, Tremelling M, Anderson C A, et al. (2007) Sequence variants in the autophagy gene IRGM and multiple other replicating loci contribute to Crohn's disease susceptibility. Nat. Genet.
31. Genome-wide association study of 14,000 cases of seven common diseases and 3,000 shared controls. Nature 447: 661-678.
32. Nicolae D L, Wen X, Voight B F, Cox N J (2006) Coverage and characteristics of the Affymetrix GeneChip Human Mapping 100K SNP set. PLoS Genet 2: e67.
33. Barrett J C, Cardon L R (2006) Evaluating coverage of genome-wide association studies. Nat Genet 38: 659-662.
34. Lin Z, Poritz L S, Li T, Byrnes K A, Wang Y, et al. (2007) Gender related association of DLG5 variant R30Q with inflammatory bowel disease (IBD) in a familial IBD registry. Gastroenterology 132: A450 (abstract).
35. Daly M J, Pearce A V, Farwell L, Fisher S A, Latiano A, et al. (2005) Association of DLG5 R30Q variant with inflammatory bowel disease. Eur J Hum Genet 13: 835-839.
36. Newman W G, Gu X, Wintle R F, Liu X, van Oene M, et al. (2006) DLG5 variants contribute to Crohn disease risk in a Canadian population. Hum Mutat 27: 353-358.
37. Yamazaki K, Takazoe M, Tanaka T, Ichimori T, Saito S, et al. (2004) Association analysis of SLC22A4, SLC22A5 and DLG5 in Japanese patients with Crohn disease. J Hum Genet 49: 664-668.
38. Lin Z, Poritz L S, Li T, Byrnes K A, Wang Y, et al. (2007) Genetic variants of DLG5 gene, E514Q, P979L, G1066G, and P1371Q in a familial inflammatory bowel disease (IBD) registry. Gastroenterology 132: A451 (abstract).
39. Weersma R K, Stokkers P C, van Bodegraven A A, van Hogezand R A, Verspaget H W, et al. (2007) A large, nationwide, case-control study for the association of DLG5, OCTN1/2 and CARD15 with Inflammatory Bowel Diseases in the Netherlands. Gastroenterology 132: A445 (abstract).
40. Russell R K, Drummond H E, Nimmo E R, Anderson N, Wilson D C, et al. (2007) The contribution of the DLG5 113A variant in early-onset inflammatory bowel disease. J Pediatr 150: 268-273.
41. de Ridder L, Weersma R K, Dijkstra G, van der Steege G, Benninga M A, et al. (2007) Genetic susceptibility has a more important role in pediatric-onset Crohn's disease than in adult-onset Crohn's disease. Inflamm Bowel Dis advance online publication, DOI: 10.1002/ibd.20171.
42. Friedrichs F, Brescianini S, Annese V, Latiano A, Berger K, et al. (2006) Evidence of transmission ratio distortion of DLG5 R30Q variant in general and implication of an association with Crohn disease in men. Hum Genet 119: 305-311.
43. Romeo S, Pennacchio L A, Fu Y, Boerwinkle E, Tybjaerg-Hansen A, et al. (2007) Population-based resequencing of ANGPTL4 uncovers variations that reduce triglycerides and increase HDL. Nat Genet 39: 513-516.
44. Noble C L, Nimmo E R, Drummond H, Smith L, Arnott I D, et al. (2005) DLG5 variants do not influence susceptibility to inflammatory bowel disease in the Scottish population. Gut 54: 1416-1420.
45. Vermeire S, Pierik M, Hlavaty T, Claessens G, van Schuerbeeck N, et al. (2005) Association of organic cation transporter risk haplotype with perianal penetrating Crohn's disease but not with susceptibility to IBD. Gastroenterology 129: 1845-1853.
46. Torok H P, Glas J, Tonenchi L, Lohse P, Muller-Myhsok B, et al. (2005) Polymorphisms in the DLG5 and OCTN cation transporter genes in Crohn's disease. Gut 54: 1421-1427.
47. Friedrichs F, Stoll M (2006) Role of discs large homolog 5. World J Gastroenterol 12: 3651-3656.
48. Matsuhashi S, Noji S, Koyama E, Myokai F, Ohuchi H, et al. (1996) New gene, nel, encoding a Mr 91 K protein with EGF-like repeats is strongly expressed in neural tissues of early stage chick embryos. Dev Dyn 207: 233-234.
49. Matsuhashi S, Noji S, Koyama E, Myokai F, Ohuchi H, et al. (1995) New gene, nel, encoding a M(r) 93 K protein with EGF-like repeats is strongly expressed in neural tissues of early stage chick embryos. Dev Dyn 203: 212-222.
50. Luce M J, Burrows P D (1999) The neuronal EGF-related genes NELL1 and NELL2 are expressed in hemopoietic cells and developmentally regulated in the B lineage. Gene 231: 121-126.
51. Kuroda S, Oyasu M, Kawakami M, Kanayama N, Tanizawa K, et al. (1999) Biochemical characterization and expression analysis of neural thrombospondin-1-like proteins NELL1 and NELL2. Biochem Biophys Res Commun 265: 79-86.
52. Kuroda S, Tanizawa K (1999) Involvement of epidermal growth factor-like domain of NELL proteins in the novel protein-protein interaction with protein kinase C. Biochem Biophys Res Commun 265: 752-757.
53. Tan K, Duquette M, Liu J H, Zhang R, Joachimiak A, et al. (2006) The structures of the thrombospondin-1 N-terminal domain and its complex with a synthetic pentameric heparin. Structure 14: 33-42.
54. Bornstein P (1995) Diversity of function is inherent in matricellular proteins: an appraisal of thrombospondin 1. J Cell Biol 130: 503-506.
55. Chen H, Herndon M E, Lawler J (2000) The cell biology of thrombospondin-1. Matrix Biol 19: 597-614.
56. Voorberg J, Fontijn R, Calafat J, Janssen H, van Mourik J A, et al. (1991) Assembly and routing of von Willebrand factor variants: the requirements for disulfide-linked dimerization reside within the carboxy-terminal 151 amino acids. J Cell Biol 113: 195-205.
57. Zhang X, Kuroda S, Carpenter D, Nishimura I, Soo C, et al. (2002) Craniosynostosis in transgenic mice overexpressing Nell-1. J Clin Invest 110: 861-870.
58. Desai J, Shannon M E, Johnson M D, Ruff D W, Hughes L A, et al. (2006) Nell1-deficient mice have reduced expression of extracellular matrix proteins causing cranial and vertebral defects. Hum Mol Genet 15: 1329-1341.
59. Abitbol V, Roux C, Chaussade S, Guillemant S, Kolta S, et al. (1995) Metabolic bone assessment in patients with inflammatory bowel disease. Gastroenterology 108: 417-422.
60. Roux C, Abitbol V, Chaussade S, Kolta S, Guillemant S, et al. (1995) Bone loss in patients with inflammatory bowel disease: a prospective study. Osteoporos Int 5: 156-160.
61. Compston J E, Judd D, Crawley E O, Evans W D, Evans C, et al. (1987) Osteoporosis in patients with inflammatory bowel disease. Gut 28: 410-415.
62. Jenkins R G, Su X, Su G, Scotton C J, Camerer E, et al. (2006) Ligation of protease-activated receptor 1 enhances alpha(v)beta6 integrin-dependent TGFbeta activation and promotes acute lung injury. J Clin Invest 116: 1606-1614.
63. Monteleone G, Del Vecchio Blanco G, Monteleone I, Fina D, Caruso R, et al. (2005) Post-transcriptional regulation of Smad7 in the gut of patients with inflammatory bowel disease. Gastroenterology 129: 1420-1429.
64. Waetzig G H, Rosenstiel P, Arlt A, Till A, Brautigam K, et al. (2005) Soluble tumor necrosis factor (TNF) receptor-1 induces apoptosis via reverse TNF signaling and autocrine transforming growth factor-beta1. Faseb J 19: 91-93.
65. Croucher P J, Mascheretti S, Hampe J, Huse K, Frenzel H, et al. (2003) Haplotype structure and association to Crohn's disease of CARD15 mutations in two ethnically divergent populations. Eur J Hum Genet 11:6-16.
66. Hampe J, Schreiber S, Shaw S H, Lau K F, Bridger S, et al. (1999) A genomewide analysis provides evidence for novel linkages in inflammatory bowel disease in a large European cohort. Am J Hum Genet 64: 808-816.
67. Lennard-Jones J E (1989) Classification of inflammatory bowel disease. Scand J Gastroenterol Suppl 170: 2-6; discussion 16-19.
68. Truelove S C, Pena A S (1976) Course and prognosis of Crohn's disease. Gut 17: 192-201.
69. Onnie C M, Fisher S A, Pattni R, Sanderson J, Forbes A, et al. (2006) Associations of allelic variants of the multidrug resistance gene (ABCB1 or MDR1) and inflammatory bowel disease and their effects on disease behavior: a case-control and meta-analysis study. Inflamm Bowel Dis 12: 263-271.
70. Hampe J, Wollstein A, Lu T, Frevel H J, Will M, et al. (2001) An integrated system for high throughput TaqMan based SNP genotyping. Bioinformatics 17: 654-655.
71. Teuber M, Koch W, Manaster C, Waechter S, Hampe J, et al. (2005) Improving quality control and workflow management in high-throughput single-nucleotide polymorphism genotyping environments. Journal of the Association for Laboratory Automation 10: 43-47.
72. Hampe J, Grebe J, Nikolaus S, Solberg C, Croucher P J, et al. (2002) Association of NOD2 (CARD 15) genotype with clinical course of Crohn's disease: a cohort study. Lancet 359: 1661-1665.
73. Weckx S, Del-Favero J, Rademakers R, Claes L, Cruts M, et al. (2005) novoSNP, a novel computational tool for sequence variation discovery. Genome Res 15: 436-442.
74. Franke A, Wollstein A, Teuber M, Wittig M, Lu T, et al. (2006) GENOMIZER: an integrated analysis system for genome-wide association data. Hum Mutat 27: 583-588.
75. Barrett J C, Fry B, Mailer J, Daly M J (2005) Haploview: analysis and visualization of LD and haplotype maps. Bioinformatics 21: 263-265.
76. Dudbridge F (2003) Pedigree disequilibrium tests for multilocus haplotypes. Genet Epidemiol 25: 115-121.
77. Stephens J C, Schneider J A, Tanguay D A, Choi J, Acharya T, et al. (2001) Haplotype variation and linkage disequilibrium in 313 human genes. Science 293: 489-493.
78. Stephens M, Donnelly P (2003) A comparison of bayesian methods for haplotype reconstruction from population genotype data. Am J Hum Genet 73: 1162-1169.
79. Waetzig G H, Seegert D, Rosenstiel P, Nikolaus S, Schreiber S (2002) p38 mitogen-activated protein kinase is activated and linked to TNF-alpha signaling in inflammatory bowel disease. J Immunol 168: 5342-5351.

TABLE 1
GENI-027/01WO

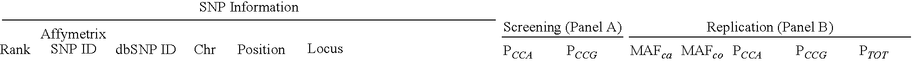

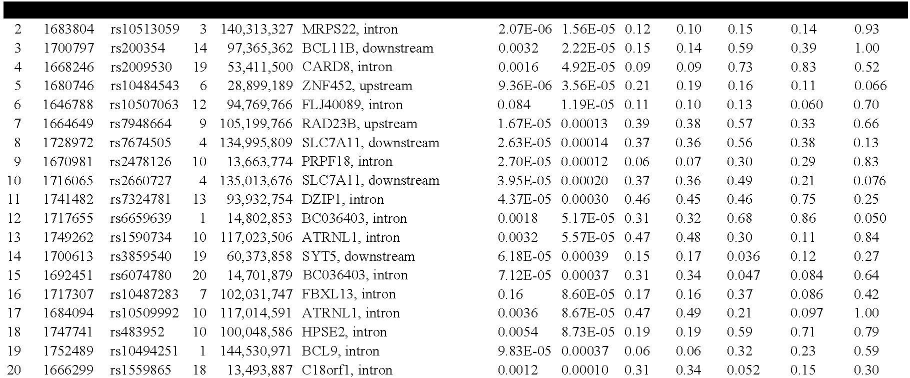

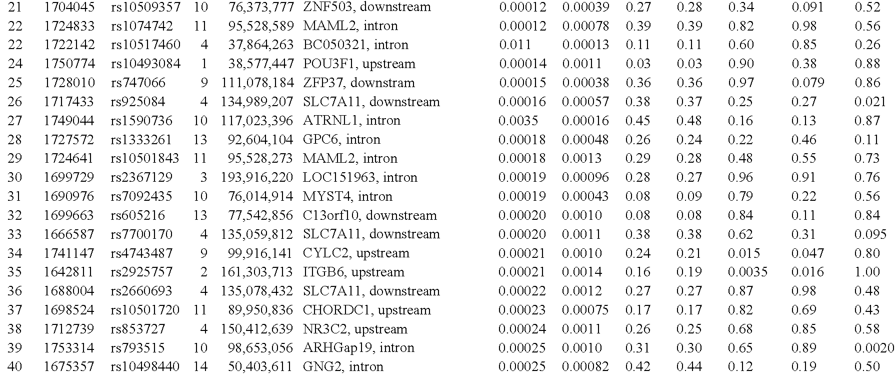

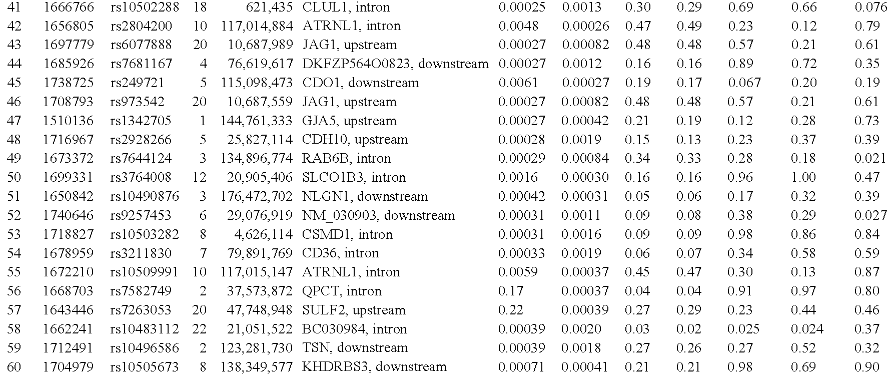

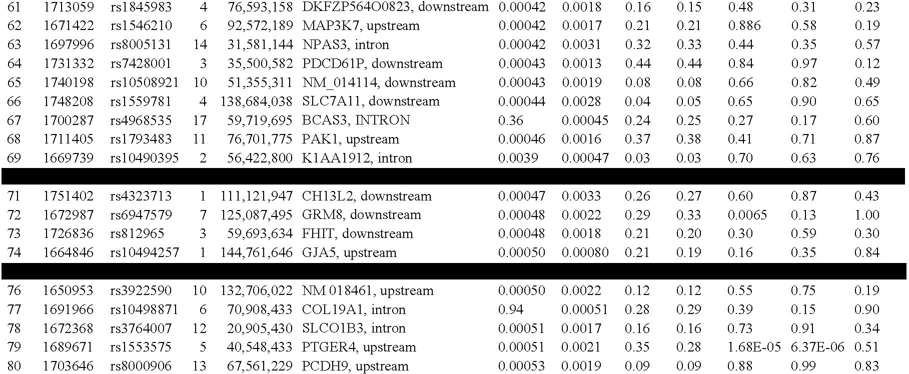

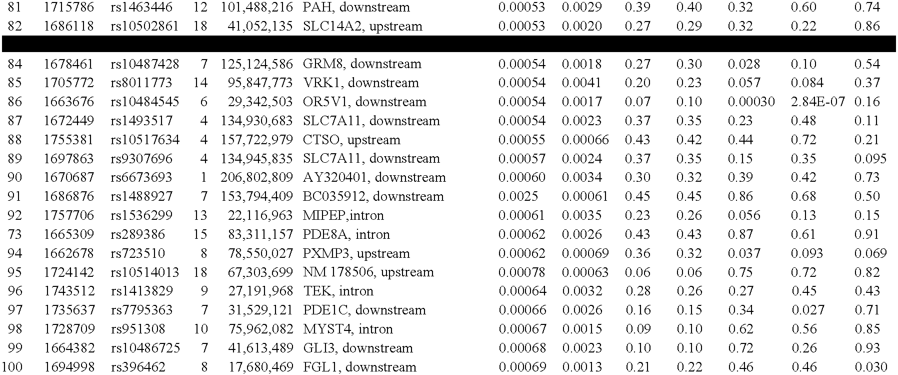

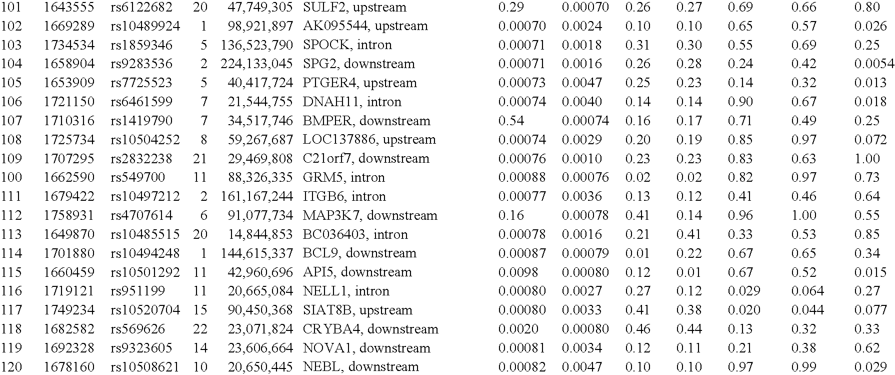

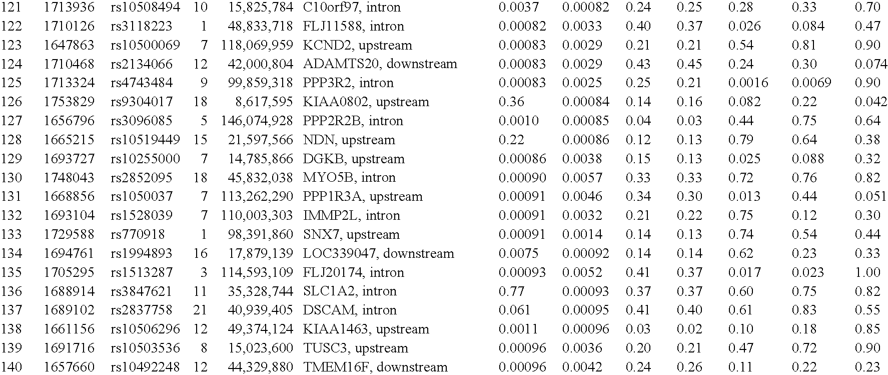

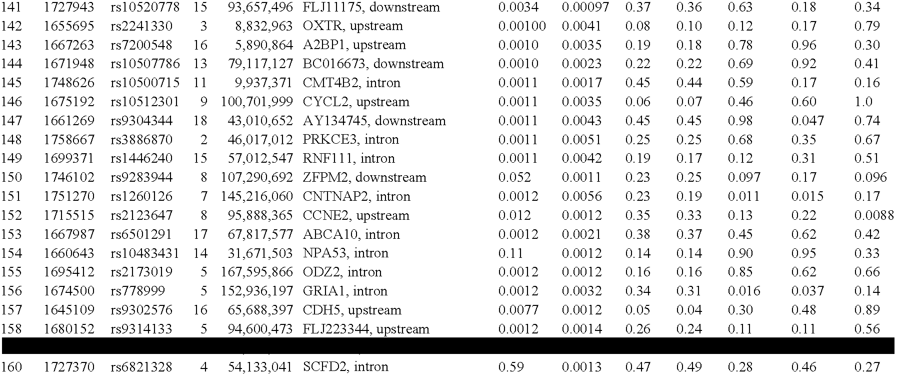

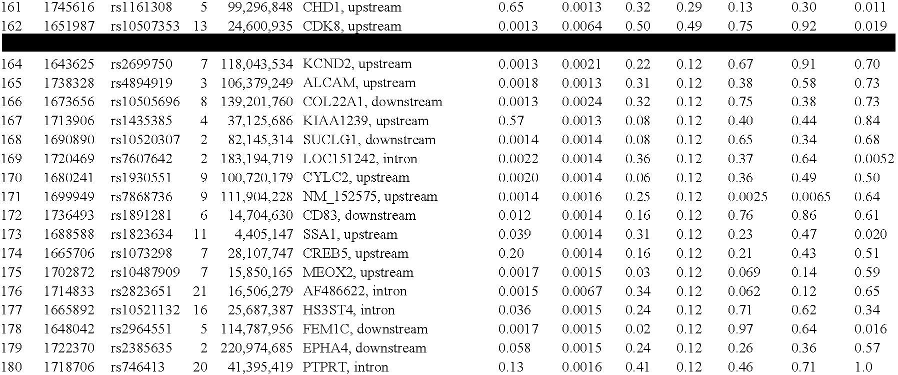

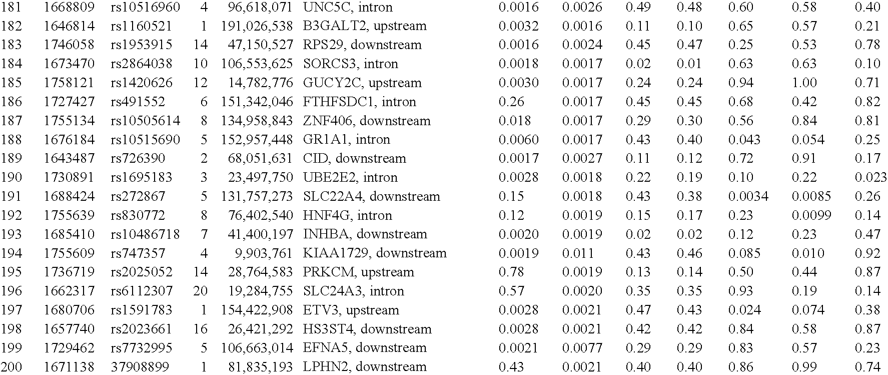

Table 2
GENI-027/01WO

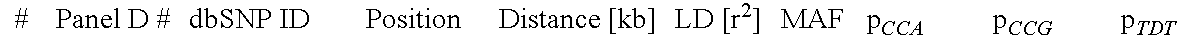

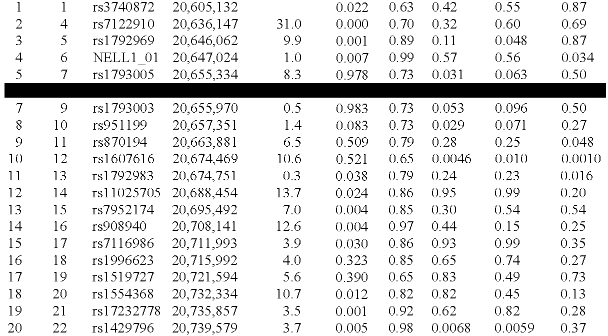

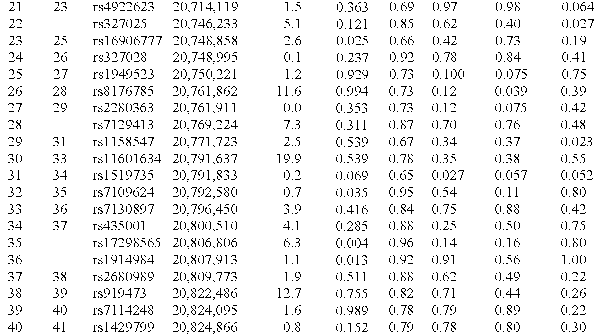

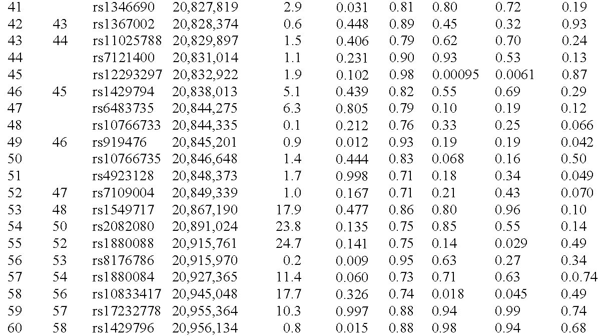

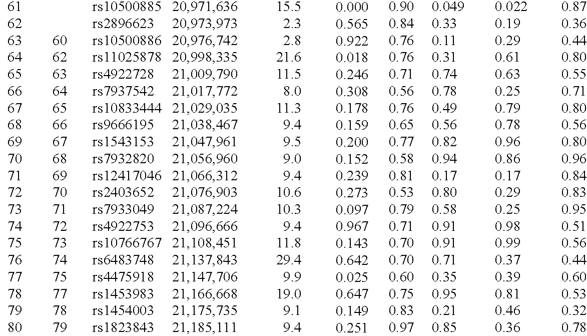

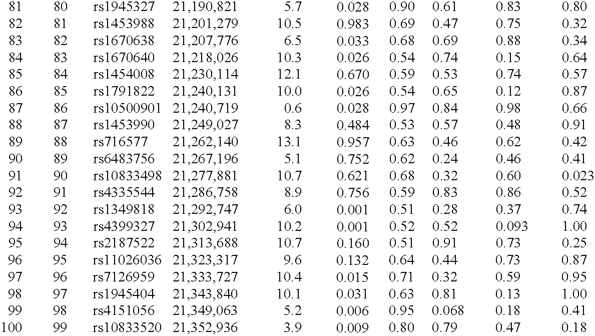

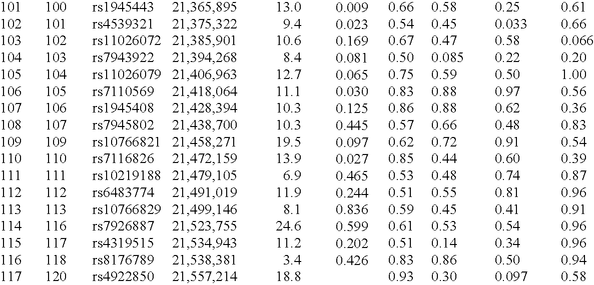

TABLE 3
GENI-027/01WO

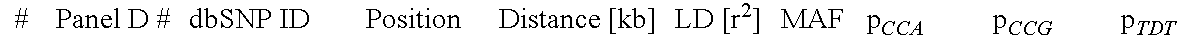

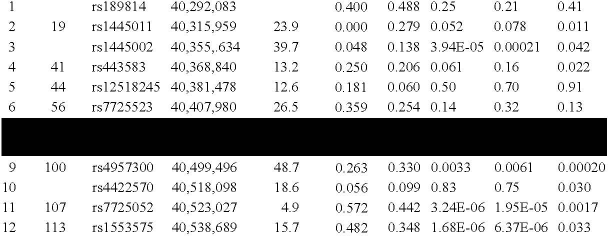

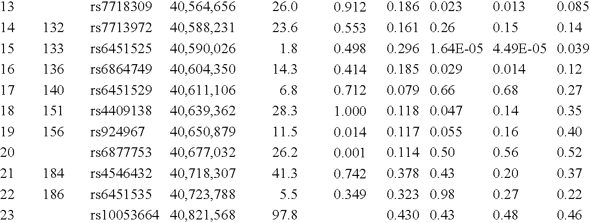

TABLE 4
GENI-027/01WO

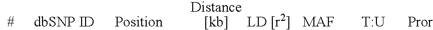

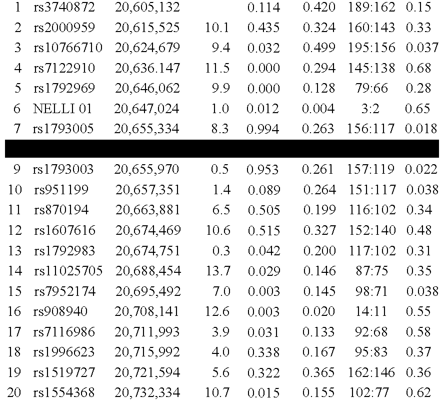

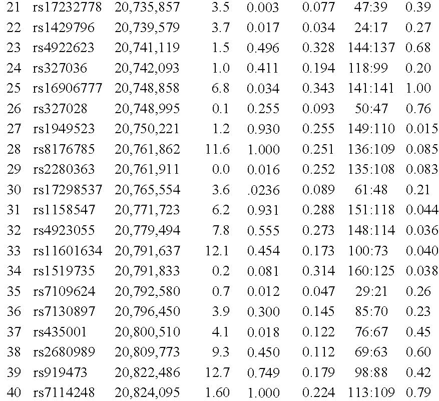

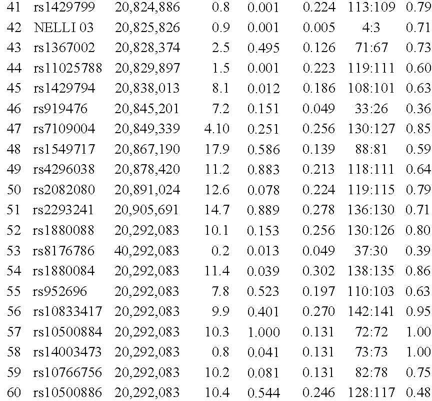

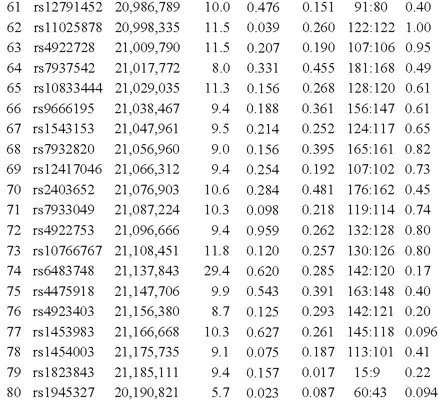

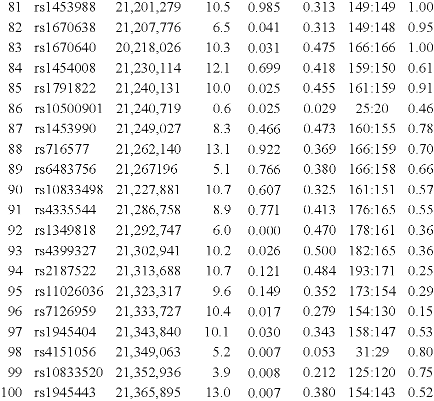

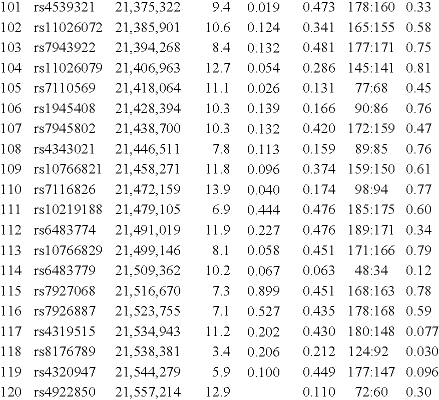

TABLE 5
GENI-027/01WO

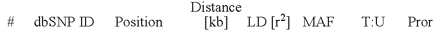

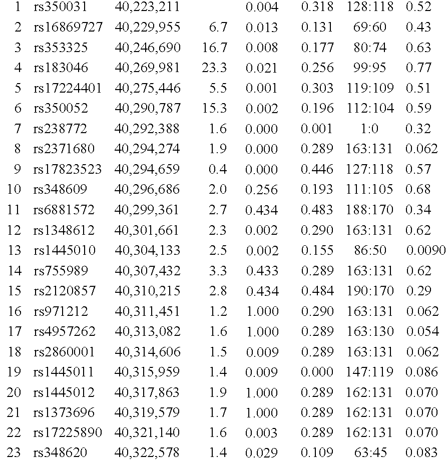

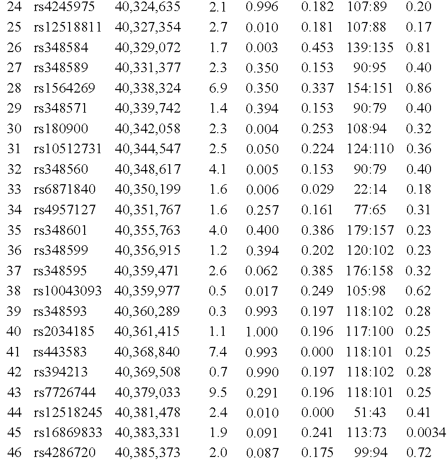

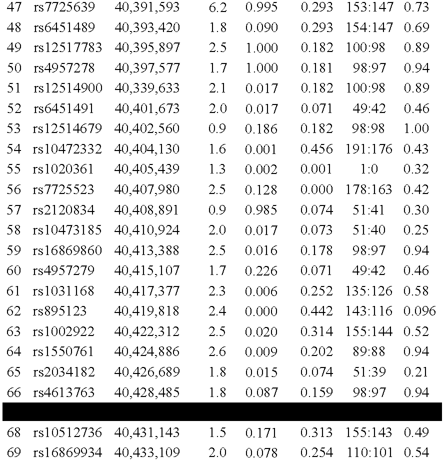

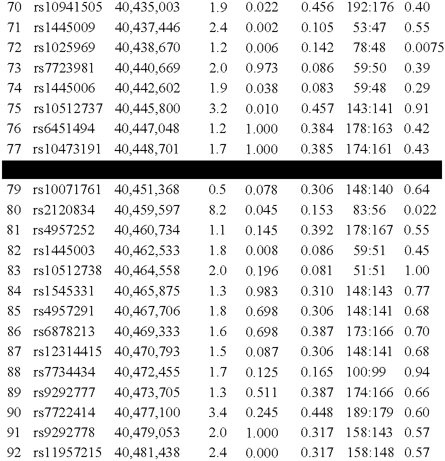

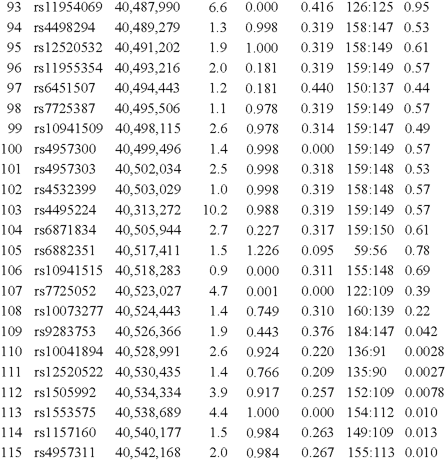

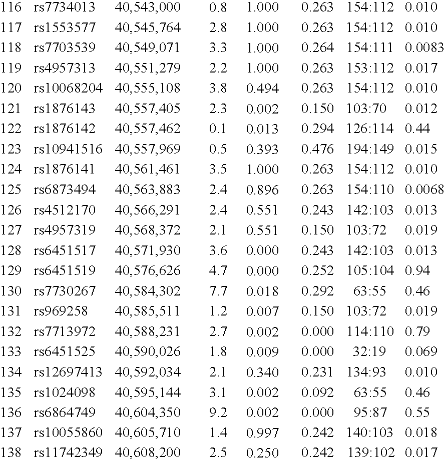

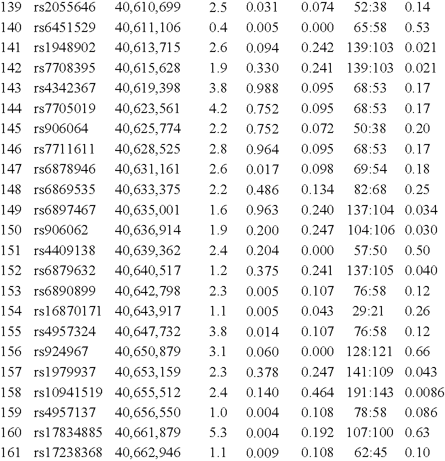

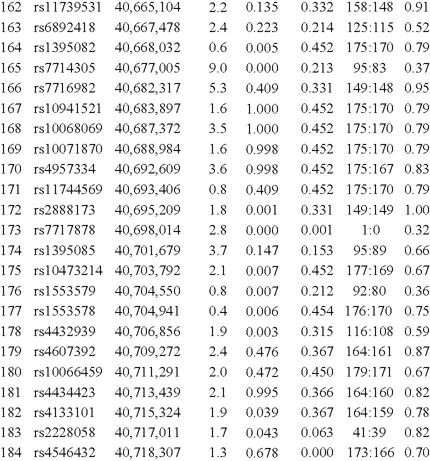

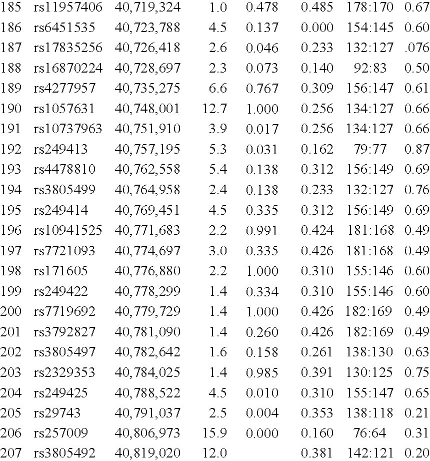

1. A method for determining inflammatory bowel disease (IBD) genotype in a patient suspected of having an IBD, or for determining a patient's susceptibility to develop an IBD, said method comprising: determining the presence or absence of one, or a combination of, single nucleotide polymorphisms (SNPs) in a biological sample from said patient, said SNP(s) being listed in any one of Tables 1-5.

2. The method of claim 1, wherein the SNP(s) are listed in any one of Tables 2-5.

3. The method of claim 1, wherein the SNP(s) is selected from the group consisting of: rs2076756, rs1992662, rs1992660, rs1793004, rs10521209, rs2631372 and combinations thereof.

4. The method of claim 1, wherein the SNP(s) is associated with a mutation in the gene encoding the nel-like 1 precursor (NELL1) in said biological sample from said patient.

5. The method of claim 4, wherein the SNP is listed in Tables 1, 2, and/or 4.

6. The method of claim 5, wherein the of SNP(s) is rs17930044.

7. The method of claim 1, wherein the SNP(s) is associated with a mutation in the 5p13.1 locus in said biological sample from said patient.

8. The method of claim 7, wherein the SNP(s) is listed in Tables 1, 3, and/or 5.

9. The method of claim 8, wherein the SNP(s) is selected from the group consisting of rs1992662 and rs19926604.

10.-12. (canceled)

13. The method of claim 1, wherein said IBD is Crohn's disease or ulcerative colitis.

14. (canceled)

15. The method of claim 4, further comprising, determining the level of expression and/or activity of NELL1 in said biological sample from said patient.

16. The method of claim 1, further comprising, determining the presence or absence of one or more of the following: a mutation in the CARD15 gene, a mutation in the DLG5 gene, a mutation in the TNFSF15 gene, a mutation in the IL23R gene, and/or a T300A mutation in the ATG16L1 gene.

17. The method of claim 4, further comprising determining the presence or absence of a mutation in the 5p13.1 locus.

18. The method of claim 17, wherein the mutation is associated with the presence or absence of SNP rs1992662 and/or rs1992660.

19.-21. (canceled)

22. A kit for determining inflammatory bowel disease (IBD) genotype in a patient suspected of having an IBD, or for determining a patient's susceptibility to develop an IBD, said kit comprising a set of nucleic acid probes and/or primers specific designed to detect two or more SNP(s) listed in any one of Tables 1-5, wherein the set of probes and/or primers consists essentially of probes and/or primers related to evaluating said IBD genotype and probes and/or primers related to assay controls.

23. The kit of claim 22, wherein the kit comprises nucleic acid probes specific for two or more SNP(s) listed in any one of Tables 2-5.

24. The kit of claim 22, wherein the kit comprises nucleic acid probes specific for two or more SNP(s) selected from the group consisting of: rs2076756, rs1992662, rs1992660, rs1793004, rs10521209, and rs2631372.

25. The kit of claim 22, wherein the kit comprises nucleic acid probes specific for each of rs2076756, rs1992662, rs1992660, rs1793004, rs10521209, and rs2631372.

26. A NELL1 polypeptide comprising at least one amino acid substitution that is associated with IBD.

27. The polypeptide of claim 26, wherein the polypeptide comprises one or more amino acid substitutions selected from Q82R, R136S, A153T or R354W.

28. A polynucleotide encoding the polypeptide of claim 26.

29. A host cell harboring the polynucleotide of claim 28.

30. An antibody specific for or raised against the polypeptide of claim 26.

31.-32. (canceled)

33. A method for identifying an agent for treating IBD, comprising contacting a NELL1 polypeptide with a test agent, and determining a change in the level of NELL1 activity as a result of the test agent.

34. The method of claim 4, wherein said patient is suffering from sarcoidosis, is suspected of having sarcoidosis, or is suffering from symptoms of sarcoidosis.

35. The method of claim 34, wherein the SNP is rs951199.
